(19) United States
(12) Patent Application Publication
(10) Pub. No.: US 2011/0117550 A1
(43) Pub. Date: May 19, 2011

(54) METHOD OF DETERMINING ANTI-HYPERTENSIVE DRUG THERAPY BY GENETIC PROFILING

(76) Inventor: Gordon S. Huggins, Bedford, MA (US)

(21) Appl. No.: 12/808,483

(22) PCT Filed: Dec. 18, 2008
(86) PCT No.: PCT/US08/13867

§ 371 Date: Jan. 19, 2011

Related U.S. Application Data
(62) Division of application No. 61/008,209, filed on Dec. 18, 2007.

(51) Int. Cl. C12Q 1/68

(52) U.S. Cl. 435/6

(57) ABSTRACT

The present invention comprises a method for determining an anti-hypertension therapy for an individual based upon the presence or absence of specific alleles affecting baseline blood pressure and sensititivity to different therapeutic formulations.

### GOVERNMENT SUPPORT

This invention was made with Government support under HL077378 (PI Michael Mendelsohn, MD) and HL069770 (Framingham Heart Study) awarded by the National Institutes of Health. The Government has certain rights in the invention.

### FIELD OF THE INVENTION

This invention relates generally to the fields of genetics and cardiovascular disease.

### BACKGROUND OF THE INVENTION

Elevated blood pressure remains the most common cardiovascular disease, contributing significant morbidity and mortality worldwide. The genetic and environmental factors responsible for cardiovascular disease are not well defined.

### SUMMARY OF THE INVENTION

The present invention comprises a method of selecting an anti-hypertensive therapy for an individual comprising the steps of identifying an individual with a hypertension condition, testing for the presence or absence of a non-synonymous substitution within the nucleic acid or amino acid sequence of the BK channel protein; and selecting a therapeutic treatment in accordance with the results of genetic testing. Substitutions that result in amino acid replacements are nonsynonymous while substitutions that do not case an amino acid replacement (such as a change in the nucleotide sequence from GGG to GGC in which both codons encode glycine) are referred to as synonymous substitutions. In one preferred embodiment, a non-synonymous substitution results from a change in the nucleotide sequence that subsequently causes a change in the amino acid sequence.

Genetic testing reveals differences in the nucleic acid or amino acid sequences that encode for a BK channel protein. The BK channel protein comprises a transcript variant of KCNMA1, KCNMB1, KCNMB2, or KCNMB3. Alternatively, or in addition, the BK channel protein comprises a protein isoform of KCNMA1, KCNMB1, KCNMB2, or KCNMB3. In a preferred embodiment, the BK channel protein comprises a transcript variant or protein isoform of KCNMB1, which is a blood vessel wall specific isoform associated with cardiovascular function. In a preferred embodiment, the BK channel protein comprises a transcript variant or protein isoform of KCNMA1, which is associated with blood pressure regulation. For example, the mutation leads to a substitution of a glutamic acid (E) residue for an amino acid residue other than glutamic acid at position 65 of the amino acid sequence encoding KCNMB1. The genetic test results in the identification of a substitution of a lysine (K) for a glutamic acid (E) residue at amino acid position 65 (E65K) in the KCNMB1 protein. Consequently, individuals who carry the E65K substitution are provided at least a beta blocker as an anti-hypertensive therapy. For example, a change in the nucleotide sequence of KCNMB1 at positions 593-595 (gag) to either “aag” to “aaa” would result in the substitution of a lysine (K) residue. In a preferred embodiment the E65K mutation results from a single polynucleotide polymorphism, or the mutation of a single nucleotide of codon 65. Thus, a mutation of the sequence “gag” to “aag” resulting in the substitution of a lysine for a glutamic acid residue, occurs in a preferred embodiment.

The anti-hypertensive therapy of the present invention is a diuretic, an angiotensin-coverting enzyme (ACE) inhibitors, an angiotensin-2 receptor antagonist, a beta blocker, a calcium channel blocker, an alpha blocker, or an antiandrenergic. Moreover, the present invention comprises a combination of one or more anti-hypertensive therapies. In an alternate embodiment, the anti-hypertensive therapy is not verapamil.

The present invention encompasses hypertension that is either primary or secondary.

Publications, U.S. patents and applications, Genbank/NCBI accession numbers, and all other references cited herein, are hereby incorporated by reference.

### BRIEF DESCRIPTION OF THE DRAWINGS

FIG. 1 is a table showing clinical characteristics of Heart SCORE and FHS subjects.

FIG. 2 is a table showing multivariate associations between E65K polymorphism and BP measurements.

FIG. 3 is a table showing odds of systolic and diastolic hypertension for E65K K-allele carriers.

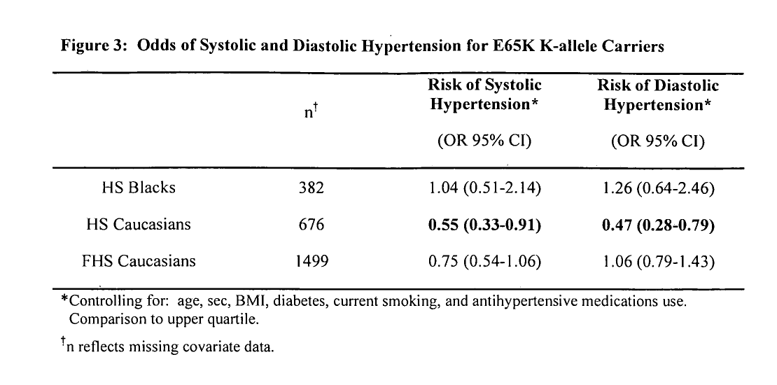

FIG. 4 is multivariate associations between E65K polymorphism and blood pressure measurements by anti-hypertensive class.

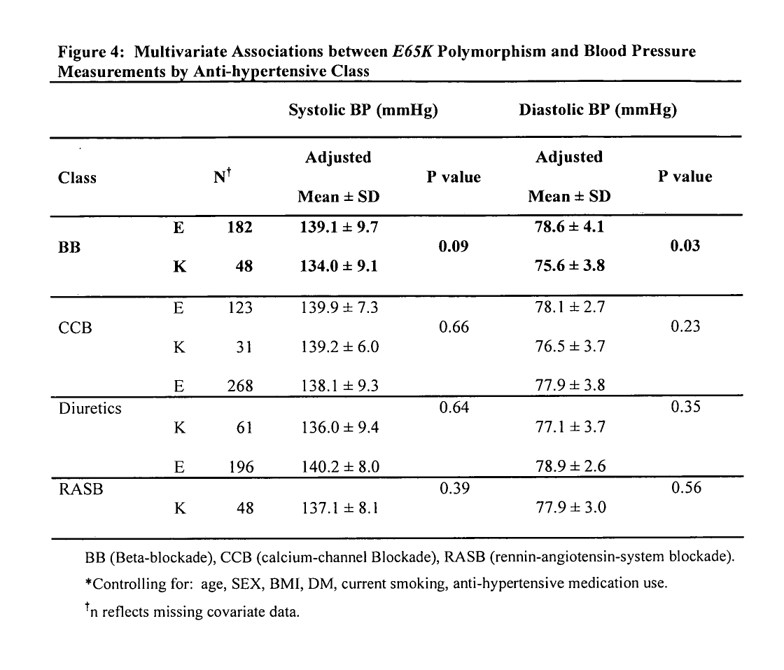

FIG. 5 is a bar graph showing adjusted-mean systolic, diastolic and pulse pressures by E65K genotype in caucasians using antihypertensive medications. Compared with E-carriers (shaded bar), K-carriers (white bar) exhibit lower adjusted systolic and diastolic pressures. No significant difference was noted in subjects not receiving therapy. Adjusted mean pressures (mmHg) for each group are shown above bar.

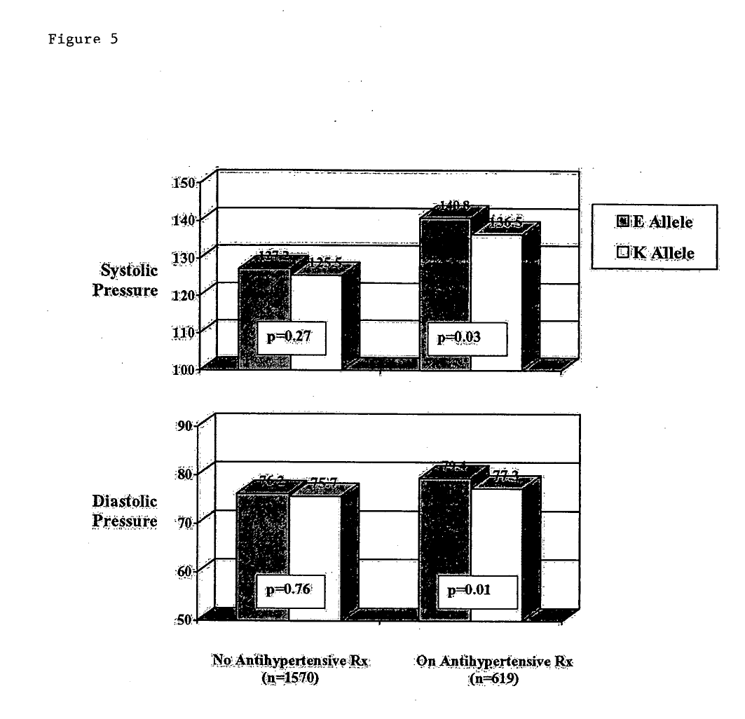

FIG. 6 is a bar graph showing adjusted-mean systolic and diastolic pressure by E65K genotype in subjects receiving beta-blockade. Results shown for entire cohort, and limited to beta-blockade use alone. Compared with E-carriers, in the presence of beta-blockade, K-carriers (white bar) exhibit lower diastolic pressure, with a trend toward significantly systolic pressure. Adjusted mean pressures (mmHg) for each group are shown above bar.

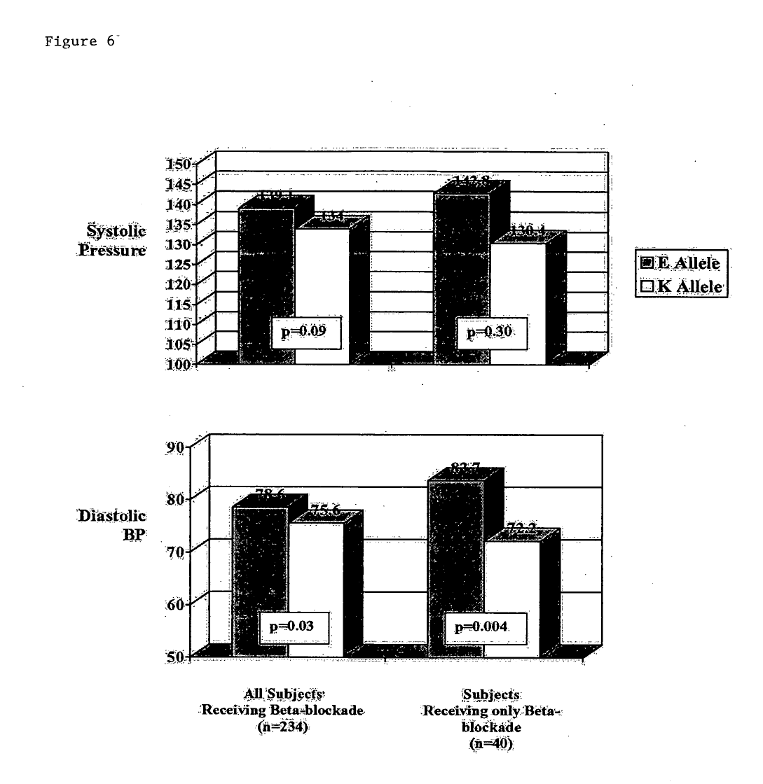

### DETAILED DESCRIPTION

Genetic polymorphisms that influence smooth muscle cell contraction and relaxation affect the response to anti-hypertensive therapy. The β subunit of the large conductance calcium-activated potassium channel contains a non-synonymous polymorphism (E65K, minor allele frequency ˜10%) that is associated with reduced diastolic pressure. Studies were carried out to determine whether E65K is associated with blood pressure in the Heart SCORE (n=1,075) and Framingham Heart Study (n=1,519) cohorts without cardiovascular disease. In Heart SCORE Caucasians (n=687), but not African-Americans (n=388), analysis of covariance revealed an association between E65K and lower systolic (E=wild type, K=substitution) (adjusted-mean mmHg (±SD) for E: 133.7 (±7.3), K 130.6 (±8.4), p=0.02) and diastolic pressures (E 79.7 (±2.6), K 77.6 (±3.1), p=0.02). In logistic regression, Caucasian K-carriers were at lower risk for elevated systolic (OR=0.55, 95% CI: 0.33-0.91) and diastolic (OR=0.47, 95% CI: 0.28-0.79) blood pressure. The protective effect of E65K was primarily evident only among subjects receiving anti-hypertensive therapy. In the Framingham Heart Study, the E65K polymorphism was not associated with blood pressure or hypertension, although a trend similar was noted. In pooled Caucasian Heart SCORE and Framingham Heart Study subjects we found that in subjects receiving beta-blockade, K-carriers had lower systolic and diastolic pressure (p=0.09, 0.03, respectively). In summary, Caucasians wild-type carriers are less responsive to beta-blockade treatment than subjects harboring the E65K allele. This discovery is consistent with carriers of the E65K allele being more responsive to beta-blocker treatment for hypertension compared with subjects that do not carry the allele. This finding indicates that E65K genetic testing is useful to determine whether beta-blocker therapy would be a preferred agent for treatment of hypertension.

Carriers of a polymorphism in the KCNMB1 gene are characterized by a greater sensitivity to beta blocker treatment in the setting of hypertension. The KCNMB1 carrier status is used to identify patients that respond favorably to beta blocker treatment. Thus, identification of this polymorphism is useful for genetic testing and the practice of personalized medicine in which patients are targeted for treatment with beta blockers as distinguished from other treatment options.

### Blood Pressure

Blood pressure, or vascular pressure, is the force exerted by circulating blood on the walls of blood vessels. The term “blood pressure” predominantly refers to arterial pressure, however, blood pressure measurements reflect the combined pressure of blood circulating through arteries, arterioles, capillaries, and veins. Arterial pressure is most commonly reported in millimetres of mercury (mmHg) and measured using a sphygmomanometer, in which changes in the height of a column of mercury reflect the circulating pressure.

Blood pressure varies with the progression through the cardiac cycle and with circadian rhythms. The term “cardiac cycle” refers the series of events that occur from the beginning of one heartbeat to the beginning of the next heartbeat. The term “heart rate” refers to the frequency of the cardiac cycle. Every heartbeat comprises three major stages: atrial systole, ventricular systole and complete cardiac diastole. The term “diastole” refers to the relaxation of the cardiac muscle.

The term “systolic” arterial pressure refers to the peak pressure and occurs near the beginning of the cardiac cycle. Conversely, the term “diastolic” arterial pressure referes to the lowest pressure and occurs during the resting phase of the cardiac cycle. The term “mean” arterial pressure is the average of the systolic and diastolic values. The term “pulse pressure” refers to the difference between the highest and lowest pressures observed during the cardiac cycle. Healthy adults, at rest, typically have a systolic score of 120 mmHg and a diastolic score of 80 mmHg. These pressures are often reported as a ratio of systolic over diastolic pressures, e.g. 120/80 mmHg. The resting pulse pressure in healthy adults is about 40 mmHg.

Individuals differ greatly in baseline, or resting, blood pressure. If the baseline blood pressure of an individual is chronically elevated above average, e.g. the individual has a blood pressure ratio of 140/90 mmHg (systolic/diastolic) or greater, he/she is diagnosed with hypertension. In the present invention, hypertension is defined by the blood pressure ratio of at least 140/90 mmHg (systolic/diastolic).

Hypertension is categorized as either primary or secondary depending on the cause. Primary hypertension is diagnosed when no specific medical cause is immediately evident. Secondary hypertension is diagnosed when hypertension occurs as a secondary effect caused by another medical condition. Individuals show variable suseptibility to primary and secondary hypertension as well as variable responses to anti-hypertensive drug therapy.

Variation in responses of individuals to anti-hypertensive drug therapy can result from a number of factors including, but not limited to, age, gender, race, diet, weight, hormone levels/balance, insulin resistance, smoking habits, fitness level, sleep/wake cycles, primary illness, medication, and stress. Body mass index (BMI, calculated by dividing the weight in kilograms by the height in meters squared), Diabetes (defined as fasting blood glucose greater than 126 mg/dL) and smoking (based on subject reporting of cigarette use) are contemplated iinfluenctial hypertensive factors.

Hypertension can be a secondary consequence of a primary illness. Exemplary primary conditions that cause secondary hypertension include, but are not limited to, renal disorders, renovascular hypertension, chronic renal failure, kidney disease, renal artery stenosis, renal segmental hypoplasia, endocrine disorders, pheochromocytoma, hyperaldosteronism (Conn's syndrome), Crushing's disease, hyperparathyroidism, acromegaly, hyperthyroidism, hypothyroidism, obstructive sleep apnea, unbalanced diet, scleroderma, neurofibromatosis, pregnancy, cancer, drugs, alcohol, nasal decongenstants, non-steroidal anti-inflammatory drugs (NSAIDs), monoamine oxidase inhibitors (MAOIs), adrenoceptor stimulants, hormonal contraception, steroid use, malformed aorta, slow pulse, aortic valve disease, coarcation of the aorta, anemia, and fever.

A major factor influencing individual susptibility to hypertension and response to treatment is genetic variation. Individuals carry mutations in genes that directly affect cellular events within blood vessel walls and, consequently, blood pressure.

### BK Channel

The BK channel regulates vascular smooth muscle polarity and, by extension, cellular contraction and relaxation. Studies on the BK channel have shown that genetic effects within the blood vessel wall can have a direct effect on blood pressure. For example, mice lacking the BK channel β1-regulatory subunit show reduced calcium-dependent channel opening, blood vessel constriction, and increased blood pressure.

The BK channel (also called a Maxi-K or slo1 channel) is a large conductance calcium-activated potassium channel. This channel mediates the efflux of potassium from the cell cytoplasm into the extracellular space when activated by changes in membrane potential and/or increases in intracellular calcium ions. Activation of the BK channel results in hyperpolarization of the cell and a decrease in cell excitability. The BK channel comprises a pore-forming alpha subunit (the ion channel) and a regulatory beta subunit.

The present invention encompasses testing for changes in the human nucleotide or amino acid sequences of the alpha and beta subunits of one or more BK channel(s).

Human KCNMB1 is encoded by the following mRNA sequence (NCBI Accession No. NM_{—}004137 and SEQ ID NO: 1; for all nucleotide sequences, the start codon, ATG, is capitalized and bolded)(For this sequence the codon encoding for amino acid position 65 is bolded:

```
      1 cagagagaag aagaaatatt tccaaatata ccaggctgat ctttcgtagt ttctcagtct
    61 tgctgtctgt tgttgtttga aatctgtttc ccacgagact ctgggaagat ggaggtgcag
  121 ggcttgggta acagctgcct tcctcagggc tgggtacctg ctctccgagt ctgtgtctcc
  181 aggctgtgtg ctcagacttg accatgtttg ggcatggagg cttgccccag atggggacca
  241 aaagctgctt ccctggagac gggcggggag gccaaacttc ctgctgaagc tctgtggcct
  301 cttttggggt gggggcgggg gtccaggcag aaagaaactg tctgctgctc aagacccaca
  361 ggacgccggg aagactaaat gatcactgcc cccagtgaat ATGgtgaaga agctggtgat
  421 ggcccagaag cggggagaga cacgagccct ttgcctgggt gtaaccatgg tggtgtgtgc
  481 cgtcatcacc tactacatcc tggtcacgac tgtgctgccc ctctaccaga aaagcgtgtg
  541 gacccaggaa tccaagtgcc acctgattga gaccaacatc agggaccagg aggagctgaa
  601 gggcaagaag gtgccccagt acccatgcct gtgggtcaac gtgtcagctg ccggcaggtg
  661 ggctgtgctg taccacacgg aggacactcg ggaccagaac cagcagtgct cctacatccc
  721 aggcagcgtg gacaattacc agacggcccg ggccgacgtg gagaaggtca gagccaaatt
  781 ccaagagcag caggtcttct actgcttctc cgcacctcgg gggaacgaaa ccagcgtcct
  841 attccagcgc ctctacgggc cccaggccct cctcttctcc ctcttctggc ccaccttcct
  901 gctgaccggt ggcctcctca ttatcgccat ggtgaagagc aaccagtacc tgtccatcct
  961 ggcggcccag aagtagagcc atccatccat gccataccac ttgtcagggc acaggggact
1021 ggctgggccc ccagggctgc tccccacttg cagcacaatg ccttctccac ctgccctccc
1081 actcttccag tccaatccac gctgtcttct gttgcaggac taacctttga gaaatccttt
1141 tgtgaagtca ttgcctgctc aagaatgtac agtggctccc caatgccttg gaggccataa
1201 ggccagccag ttctagctct ctattacctg tccccactca actgactcat acctgtttcc
1261 ggctgcatca ctatgtgccc cacagagaac gatgatcgtc acctctgtgc ctgagttctc
1321 cctgttgtct caaagcggta cccatcctcc cccagaagct gtccccagcg agcctccctt
1381 ctttgtttga attctctaat aagagcaaca gcagctccca tttctagaac acatttacag
1441 tattactatt ttctaggata taaagtgcca tatatatttt taattccaat attaataaat
1501 gtatgccaaa caacaata
```

Human KCNMB1 is encoded by the following polypeptide sequence (NCBI Accession No. NP_{—}004128.1 and SEQ ID NO: 2)(For this sequence position 65 is bolded):

MVKKLVMAQKRGETRALCLGVTMVVCAVITYYILVTTVLPLYQKSVWTQ
ESKCHLIETNIRDQEELKGKKVPQYPCLWVNVSAAGRWAVLYHTEDTRD
QNQQCSYIPGSVDNYQTARADVEKVRAKFQEQQVFYCFSAPRGNETSVL
FQRLYGPQALLFSLFWPTFLLTGGLLIIAMVKSNQYLSILAAQK

Human KCNMB2, transcript variant 1, is encoded by the following mRNA sequence (NCBI Accession No. NM_{—}181361 and SEQ ID NO: 3):

```
      1 agtatgaagc agctttaacg gagctccaga taactaattt caagcatggc tgtctagcgt
    61 gcctgtcact cctattgtcc ttccaaactc tttcagacat tttgagcagg gagtattaaa
  121 gctatgagtt agaaagggtt gtgacattaa tggtccacaa aggctttagg cacaagaggt
  181 aatgatgatt accggtggct atttggagga ctgcaccgga tcgctgtcat taaaaattaa
  241 gcgtggcttt tgaggaagat gtgacaacta ccggaggtct ttttgccatt cctccaggac
  301 atccaccata aggaaaggag accctggacc aacattctct aagATGttta tatggaccag
  361 tggccggacc tcttcatctt atagacatga tgaaaaaaga aatatttacc agaaaatcag
  421 ggaccatgac ctcctggaca aaaggaaaac agtcacagca ctgaaggcag gagaggaccg
  481 agctattctc ctgggactgg ctatgatggt gtgctccatc atgatgtatt ttctgctggg
  541 aatcacactc ctgcgctcat acatgcagag cgtgtggacc gaagagtctc aatgcacctt
  601 gctgaatgcg tccatcacgg aaacatttaa ttgctccttc agctgtggtc cagactgctg
  661 gaaactttct cagtacccct gcctccaggt gtacgttaac ctgacttctt ccggggaaaa
  721 gctcctcctc taccacacag aagagacaat aaaaatcaat cagaagtgct cctatatacc
  781 taaatgtgga aaaaattttg aagaatccat gtccctggtg aatgttgtca tggaaaactt
  841 caggaagtat caacacttct cctgctattc tgacccagaa ggaaaccaga agagtgttat
  901 cctaacaaaa ctctacagtt ccaacgtgct gttccattca ctcttctggc caacctgtat
  961 gatggctggg ggtgtggcaa ttgttgccat ggtgaaactt acacagtacc tctccctact
1021 atgtgagagg atccaacgga tcaatagata aatgcaaaaa tggataaaat aatttttgtt
1081 aaagctcaaa tactgttttc tttcattctt caccaaagaa ccttaagttt gtaacgtgca
1141 gtctgttatg agttccctaa tatattctta tatgtagagc aataatgcaa aagctgttct
1201 atatgcaaac atgatgtctt tattattcag gagaataaat aactgttttg tgttggttgg
1261 tggttttcat aatcttattt ctgtactgga actagtactt tcttctctca ttccgccaaa
1321 acagggctca gttattcatt tgccaagctt cgtggaggaa tgtaggtgac atcaatgtga
1381 taaagtctgt gttctgagtt gtcagatctc ttgaagacaa tatttttcat cacttattgt
1441 ttactaaagc tacagccaaa aatatttttt tttcttattc taaactgagc cctatagcaa
1501 gtgaagggac cagatttcct aattaaagga agttaggtac ttttcttgta ttttttacca
1561 tatcactgta aagaagaggg gaaacccagc cagctacttt ttttcatcac tttttattca
1621 taacttcaga tttgtaaaac taatttccaa aatataagct gttttcatta gccagttcta
1681 taatatcttc ctgtgattta tgtagaaaat gaacacaccc cttttccatt taagaccctg
1741 ctactgtgtg aagagatgat acttacaagg agtgtcatta cctgtgagct gactgaatgt
1801 tggtaggtgc tccattacaa tccaggaaag tctgtgttac tgatatttgt gtggaaatct
1861 ttatttcact tcaatttaac cattagatgg taaaattaag atgctacttg ttggtaaaaa
1921 ttggtggact ggtttcaatg ggtaaatgtg ttgtggcaaa ttaatgtgtt ggaatattgc
1981 tctttgtgaa tttgtgctta agtcaatgaa tgtgtagtat ctccttctga caagcattcc
2041 ctattgggat tttaaagcta tgtgcacaga atattagtct cttctacatg ttttattttt
2101 ctatttataa ttcccttttt tgttgttata ttttatacac agaatagatc ttttttctaa
2161 cacatatttg aactgaataa cagacttaaa gaaagccttt gttcacattg ctatttactt
2221 ttgtgtttgg gggaaaatac gagggattga ttttaaataa aaaacattcc atctttcatt
2281 taatatcaat atcaaaagaa gaagacaaac atctatcttt ctcatctata tttaagtacc
2341 tttttgtaat gtagtatcaa agttttttag gtaatgcaaa attttacaaa tcatttgtgg
2401 aatgaatggt aaaactaatc tgatgaaatg gaaaattatt ctgcaatatt gtaattcata
2461 gtttgacttt tcataagcaa ataaatccct aggatgtaat caggacttca aatgtgtaat
2521 taaatttttt taaaaaaaat cta
```

Human KCNMB2 is encoded by the following polypeptide sequence (NCBI Accession No. NP_{—}852006.1 and SEQ ID NO: 4):

MFIWTSGRTSSSYRHDEKRNIYQKIRDHDLLDKRKTVTALKAGEDRAIL
LGLAMMVCSIMMYFLLGITURSYMQSVWTEESQCTLLNASITETFNCSF
SCGPDCWKLSQYPCLQVYVNLTSSGEKLLLYHTEETIKINQKCSYIPKC
GKNFEESMSLVNVVMENFRKYQHFSCYSDPEGNQKSVILTKLYSSNVLF
HSLFWPTCMMAGGVAIVAMVKLTQYLSLLCERIQRINR

Human KCNMB2, transcript variant 2, is encoded by the following mRNA sequence (NCBI Accession No. NM_{—}181361 and SEQ ID NO: 5):

```
      1 gctgggcacc gttctgtttt ctttcttttc ttaatcctat ccaagtatgc agtacgctct
    61 tgggtcgtct catgagaccc aggggcatgt tggaaagaac tgagagaaag agcaacaaag
  121 cggcgagtgg tgtgagaggg cagcacgcgc tgtggggccc ttccagagaa atgtactgaa
  181 aaagtctacg caatgtctgg gatttgctaa acaatacctg gaaagcagac aggtcttttt
  241 gccattcctc caggacatcc accataagga aaggagaccc tggaccaaca ttctctaagA
  301 TGtttatatg gaccagtggc cggacctctt catcttatag acatgatgaa aaaagaaata
  361 tttaccagaa aatcagggac catgacctcc tggacaaaag gaaaacagtc acagcactga
  421 aggcaggaga ggaccgagct attctcctgg gactggctat gatggtgtgc tccatcatga
  481 tgtattttct gctgggaatc acactcctgc gctcatacat gcagagcgtg tggaccgaag
  541 agtctcaatg caccttgctg aatgcgtcca tcacggaaac atttaattgc tccttcagct
  601 gtggtccaga ctgctggaaa ctttctcagt acccctgcct ccaggtgtac gttaacctga
  661 cttcttccgg ggaaaagctc ctcctctacc acacagaaga gacaataaaa atcaatcaga
  721 agtgctccta tatacctaaa tgtggaaaaa attttgaaga atccatgtcc ctggtgaatg
  781 ttgtcatgga aaacttcagg aagtatcaac acttctcctg ctattctgac ccagaaggaa
  841 accagaagag tgttatccta acaaaactct acagttccaa cgtgctgttc cattcactct
  901 tctggccaac ctgtatgatg gctgggggtg tggcaattgt tgccatggtg aaacttacac
  961 agtacctctc cctactatgt gagaggatcc aacggatcaa tagataaatg caaaaatgga
1021 taaaataatt tttgttaaag ctcaaatact gttttctttc attcttcacc aaagaacctt
1081 aagtttgtaa cgtgcagtct gttatgagtt ccctaatata ttcttatatg tagagcaata
1141 atgcaaaagc tgttctatat gcaaacatga tgtctttatt attcaggaga ataaataact
1201 gttttgtgtt ggttggtggt tttcataatc ttatttctgt actggaacta gtactttctt
1261 ctctcattcc gccaaaacag ggctcagtta ttcatttgcc aagcttcgtg gaggaatgta
1321 ggtgacatca atgtgataaa gtctgtgttc tgagttgtca gatctcttga agacaatatt
1381 tttcatcact tattgtttac taaagctaca gccaaaaata tttttttttc ttattctaaa
1441 ctgagcccta tagcaagtga agggaccaga tttcctaatt aaaggaagtt aggtactttt
1501 cttgtatttt ttaccatatc actgtaaaga agaggggaaa cccagccagc tacttttttt
1561 catcactttt tattcataac ttcagatttg taaaactaat ttccaaaata taagctgttt
1621 tcattagcca gttctataat atcttcctgt gatttatgta gaaaatgaac acaccccttt
1681 tccatttaag accctgctac tgtgtgaaga gatgatactt acaaggagtg tcattacctg
1741 tgagctgact gaatgttggt aggtgctcca ttacaatcca ggaaagtctg tgttactgat
1801 atttgtgtgg aaatctttat ttcacttcaa tttaaccatt agatggtaaa attaagatgc
1861 tacttgttgg taaaaattgg tggactggtt tcaatgggta aatgtgttgt ggcaaattaa
1921 tgtgttggaa tattgctctt tgtgaatttg tgcttaagtc aatgaatgtg tagtatctcc
1981 ttctgacaag cattccctat tgggatttta aagctatgtg cacagaatat tagtctcttc
2041 tacatgtttt atttttctat ttataattcc cttttttgtt gttatatttt atacacagaa
2101 tagatctttt ttctaacaca tatttgaact gaataacaga cttaaagaaa gcctttgttc
2161 acattgctat ttacttttgt gtttggggga aaatacgagg gattgatttt aaataaaaaa
2221 cattccatct ttcatttaat atcaatatca aaagaagaag acaaacatct atctttctca
2281 tctatattta agtacctttt tgtaatgtag tatcaaagtt ttttaggtaa tgcaaaattt
2341 tacaaatcat ttgtggaatg aatggtaaaa ctaatctgat gaaatggaaa attattctgc
2401 aatattgtaa ttcatagttt gacttttcat aagcaaataa atccctagga tgtaatcagg
2461 acttcaaatg tgtaattaaa tttttttaaa aaaaatcta
```

Human KCNMB2 is encoded by the following polypeptide sequence (NCBI Accession No. NP_{—}005823.1 and SEQ ID NO: 6):

MFIWTSGRTSSSYRHDEKRNIYQKIRDHDLLDKRKTVTALKAGEDRAIL
LGLAMMVCSIMMYFLLGITLLRSYMQSVWTEESQCTLLNASITETFNCS
FSCGPDCWKLSQYPCLQVYVNLTSSGEKLLLYHTEETIKINQKCSYIPK
CGKNFEESMSLVNVVMENFRKYQHFSCYSDPEGNQKSVILTKLYSSNVL
FHSLFWPTCMMAGGVAIVAMVKLTQYLSLLCERIQRINR

Human KCNMB3, transcript variant 1, is encoded by the following mRNA sequence (NCBI Accession No. NM_{—}171828 and SEQ ID NO: 7):

```
      1 gctcccggct gccgaggcgg aaacacaggt gatgaggtgg cggcaagcac agtgcaaaga
    61 gagagaagca gcttcggctg cagcaaacca cgcaggtcct tcttgatcat ctagaactga
  121 ccgctccgcc ttgccaggag tctgcagaac cacgtggcta gcctgcctga agttctcacc
  181 tctccaggaa ggcggggggc ttctaatggc tgcagctgcg ctgggggctg ggggctcccg
  241 ctgggactcc acttccgtgg atgtctaagc ttcacctttc ttgcgcccgc aggggcatga
  301 ctcaggtgaa agggagccat tttctcagac ccctggcctc ATGcagccct tcagcatccc
  361 cgtgcaaatc acacttcagg gcagccggag gcgccagggg aggacagcct ttcctgcctc
  421 agggaagaag agagagacag actacagtga tggagaccca ctagatgtgc acaagaggct
  481 gccatccagt gctggagagg accgagccgt gatgctgggg tttgccatga tgggcttctc
  541 agtcctaatg ttcttcttgc tcggaacaac cattctaaag ccttttatgc tcagcattca
  601 gagagaagaa tcgacctgca ctgccatcca cacagatatc atggacgact ggctggactg
  661 tgccttcacc tgtggtgtgc actgccacgg tcaggggaag tacccgtgtc ttcaggtgtt
  721 tgtgaacctc agccatccag gtcagaaagc tctcctacat tataatgaag aggctgtcca
  781 gataaatccc aagtgctttt acacacctaa gtgccaccaa gatagaaatg atttgctcaa
  841 cagtgctctg gacataaaag aattcttcga tcacaaaaat ggaaccccct tttcatgctt
  901 ctacagtcca gccagccaat ctgaagatgt cattcttata aaaaagtatg accaaatggc
  961 tatcttccac tgtttatttt ggccttcact gactctgcta ggtggtgccc tgattgttgg
1021 catggtgaga ttaacacaac acctgtcctt actgtgtgaa aaatatagca ctgtagtcag
1081 agatgaggta ggtggaaaag taccttatat agaacagcat cagttcaaac tgtgcattat
1141 gaggaggagc aaaggaagag cagagaaatc ttaagacggt ggccaaatta aagtgctggc
1201 cttcagatgt ctgtgatttc tgcaactgag gacctaatta tgcctgtctg caaactaata
1261 atgtaaaagg ta
```

Human KCNMB3, transcript variant 1, is encoded by the following polypeptide sequence (NCBI Accession No. NP_{—}741979.1 and SEQ ID NO: 8):

MQPFSIPVQITLQGSRRRQGRTAFPASGKKRETDYSDGDPLDVHKRLP
SSAGEDRAVMLGFAMMGFSVLMFFLLGTTILKPFMLSIQREESTCTAI
HTDIMDDWLDCAFTCGVHCHGQGKYPCLQVFVNLSHPGQKALLHYNEE
AVQINPKCFYTPKCHQDRNDLLNSALDIKEFFDHKNGTPFSCFYSPAS
QSEDVILIKKYDQMAIFHCLFWPSLTLLGGALIVGMVRLTQHLSLLCE
KYSTVVRDEVGGKVPYIEQHQFKLCIMRRSKGRAEKS

Human KCNMB3, transcript variant 2, is encoded by the following mRNA sequence (NCBI Accession No. NM_{—}171829 and SEQ ID NO: 9):

| | |
|---|---|
| 1 | aagagaaaga acaagaaaaa gaaaaagaag aggaaaaaat ccccagtacc catagaaacc |
| 61 | cttaaagatg tttaaaaaga gttaacttat cagaacacag atttaagtga aattaaggaa |
| 121 | gaagagcagg taaagtctac tgacagaaag tcagcagtgg aagcccaaaa cgaggtgact |
| 181 | gaaaatccaa aacagaaaat tgcagcagaa agcagtgaaa atgttgattg tccagagaat |
| 241 | cctaaaatga agttggatgg aaaacttgac caagaaggca atgatgtaaa aacagcagct |
| 301 | gaggaggtac tagctggtag agacacatta gattttgagg atgtcacagt tcaatcatca |
| 361 | ggcccgaggg ctggtggtga agaattagat gaaggtgttg caaaagataa tgctaaaata |
| 421 | gctggtgcca cttaaagcaa tcctgaagaa ccagagagcg aagatgcaga tcactgcacc |
| 481 | gtacccaaaa atgaaagtcc ctcacaggac attagtgatg cctgtgaagc agaaagtaca |
| 541 | gagaggtgtg ggatgtcaga acatccaagt cagaccatca ggaaagcttt agacagcaat |
| 601 | agcctaaaaa accatgactt gttggcacca ggaggagagc cgggggactt caatccagaa |
| 661 | agcagagaag ataccagagg agggaacgag aagggcaaaa gcaaagaaga ccgtaccatg |
| 721 | tcctaagctg aggcaggcgg caggcgtggt gcacaggaag tctgagtgtg aggggctctt |
| 781 | ttctctccac tgccaATGac agcctttcct gcctcaggga agaagagaga gacagactac |
| 841 | agtgatggag acccactaga tgtgcacaag aggctgccat ccagtgctgg agaggaccga |
| 901 | gccgtgatgc tggggtttgc catgatgggc ttctcagtcc taatgttctt cttgctcgga |
| 961 | acaaccattc taaagccttt tatgctcagc attcagagag aagaatcgac ctgcactgcc |
| 1021 | atccacacag atatcatgga cgactggctg gactgtgcct tcacctgtgg tgtgcactgc |
| 1081 | cacggtcagg ggaagtaccc gtgtcttcag gtgtttgtga acctcagcca tccaggtcag |
| 1141 | aaagctctcc tacattataa tgaagaggct gtccagataa atcccaagtg cttttacaca |
| 1201 | cctaagtgcc accaagatag aaatgatttg ctcaacagtg ctctggacat aaaagaattc |
| 1261 | ttcgatcaca aaaatggaac ccccttttca tgcttctaca gtccagccag ccaatctgaa |
| 1321 | gatgtcattc ttataaaaaa gtatgaccaa atggctatct tccactgttt attttggcct |
| 1381 | tcactgactc tgctaggtgg tgccctgatt gttggcatgg tgagattaac acaacacctg |
| 1441 | tccttactgt gtgaaaaata tagcactgta gtcagagatg aggtaggtgg aaaagtacct |
| 1501 | tatatagaac agcatcagtt caaactgtgc attatgagga ggagcaaagg aagagcagag |
| 1561 | aaatcttaag acggtggcca aattaaagtg ctggccttca gatgtctgtg atttctgcaa |
| 1621 | ctgaggacct aattatgcct gtctgcaaac taataatgta aaaggtaata attaaagtat |
| 1681 | catattttca tgtgggaaaa aatttaaaa |

Human KCNMB3, transcript variant 2, is encoded by the following polypeptide sequence (NCBI Accession No. NP_{—}741980.1 and SEQ ID NO: 10):

MTAFPASGKKRETDYSDGDPLDVHKRLPSSAGEDRAVMLGFAMMGFSV
LMFFLLGTTILKPFMLSIQREESTCTAIHTDIMDDWLDCAFTCGVHCH
GQGKYPCLQVFVNLSHPGQKALLHYNEEAVQINPKCFYTPKCHQDRND
LLNSALDIKEFFDHKNGTPFSCFYSPASQSEDVILIKKYDQMAIFHCL
FWPSLTLLGGALIVGMVRLTQHLSLLCEKYSTVVRDEVGGKVPYIEQH
QFKLCIMRRSKGRAEKS

Human KCNMB3, transcript variant 3, is encoded by the following mRNA sequence (NCBI Accession No. NM_{—}171830 and SEQ ID NO: 11):

| | |
|---|---|
| 1 | cccagctact agggaggctg aggcaggaga atcgcttgaa cctgggaggc ggaggaggtt |
| 61 | gcagtgaact gagatcgtac ccagcctggg caacagtgcg aggttccgtc tcaaaaaaaa |
| 121 | ccaaaaaaca caaaaacaaa aaacgacaga gaaggccaaa caaaacacat ctgtgggctg |
| 181 | gatgccgcca tgcccaccgg tttgcgacct ttgtgttgga ctcttctgtt caccagacac |
| 241 | cctgccctgc gagaatgtat ctcatccttt gctggagcag gtttgcaggc acagtggaga |
| 301 | gaggagagaa gaaatgaagg gacacttatg cagaaccatg agtggccaga gaggaggaga |
| 361 | aggagggtga gaggagcaaa gaagccatga caacttcata attctgagtg gactgggcag |
| 421 | tggccagaaa ttctggtggt ggatatgctg cctttccaac aggtgaatat gaaagaataa |
| 481 | gtcaaaccct gttcaggacg ctgttaattc caaatgtgaa ctttttgagt cattctttcc |
| 541 | atgtggaatt caaaggagaa tgtaaacaaa ttttcaggag ggacgtgcaa tatccctgaa |
| 601 | agataacaaa gttcgtaaca cttatttaca tacaacattc tctagttatt gattaaacag |
| 661 | atctctacag acttgcatga ggcaacattt cttaggcttg tttgctacaa tatctttaaa |
| 721 | aatacttgat tacacatcac tttagcttat ttagatggac ttttcaccaa gctctgaact |
| 781 | gggatttcat tttgttgcat tcatcctgct cacgagacac aggtaggcag caaatgagat |
| 841 | tatccctcca gtccccatgg attggaaATG ttcccccttc tttatgagct cactgcagta |
| 901 | tctccttctc cctttcccca aaggacagcc tttcctgcct cagggaagaa gagagagaca |
| 961 | gactacagtg atggagaccc actagatgtg cacaagaggc tgccatccag tgctggagag |
| 1021 | gaccgagccg tgatgctggg gtttgccatg atgggcttct cagtcctaat gttcttcttg |
| 1081 | ctcggaacaa ccattctaaa gccttttatg ctcagcattc agagagaaga atcgacctgc |
| 1141 | actgccatcc acacagatat catggacgac tggctggact gtgccttcac ctgtggtgtg |
| 1201 | cactgccacg gtcaggggaa gtacccgtgt cttcaggtgt ttgtgaacct cagccatcca |
| 1261 | ggtcagaaag ctctcctaca ttataatgaa gaggctgtcc agataaatcc caagtgcttt |
| 1321 | tacacaccta agtgccacca agatagaaat gatttgctca acagtgctct ggacataaaa |
| 1381 | gaattcttcg atcacaaaaa tggaaccccc ttttcatgct tctacagtcc agccagccaa |
| 1441 | tctgaagatg tcattcttat aaaaaagtat gaccaaatgg ctatcttcca ctgtttattt |
| 1501 | tggccttcac tgactctgct aggtggtgcc ctgattgttg gcatggtgag attaacacaa |
| 1561 | cacctgtcct tactgtgtga aaaatatagc actgtagtca gagatgaggt aggtggaaaa |
| 1621 | gtaccttata tagaacagca tcagttcaaa ctgtgcatta tgaggaggag caaaggaaga |
| 1681 | gcagagaaat cttaagacgg tggccaaatt aaagtgctgg ccttcagatg tctgtgattt |
| 1741 | ctgcaactga ggacctaatt atgcctgtct gcaaactaat aatgtaaaag gtaataatta |
| 1801 | aagtatcata ttttcatgtg ggaaaaaatt taaaa |

Human KCNMB3, transcript variant 3, is encoded by the following polypeptide sequence (NCBI Accession No. NP_{—}741981.1 and SEQ ID NO: 12):

MFPLLYELTAVSPSPFPQRTAFPASGKKRETDYSDGDPLDVHKRLPSS
AGEDRAVMLGFAMMGFSVLMFFLLGTTILKPFMLSIQREESTCTAIHT
DIMDDWLDCAFTCGVHCHGQGKYPCLQVFVNLSHPGQKALLHYNEEAV
QINPKCFYTPKCHQDRNDLLNSALDIKEFFDHKNGTPFSCFYSPASQS
EDVILIKKYDQMAIFHCLFWPSLTLLGGALIVGMVRLTQHLSLLCEKY
STVVRDEVGGKVPYIEQHQFKLCIMRRSKGRAEKS

Human KCNMB3, transcript variant 4, is encoded by the following mRNA sequence (NCBI Accession No. NM_{—}014407 and SEQ ID NO: 13):

| | |
|---|---|
| 1 | tgccctgcga gaatgtatct catcctttgc tggagcaggt ttgcaggcac agtggagaga |
| 61 | ggagagaaga aatgaaggga cacttatgca gaaccatgag tggccagaga ggaggagaag |
| 121 | gagggtgaga ggagcaaaga agccatgaca acttcataat tctgagtgga ctgggcagtg |
| 181 | gccagaaatt ctggtggtgg atatgctgcc tttccaacag gtgaatatga aagaataagt |
| 241 | caaaccctgt tcaggacgct gttaattcca aatgtgaact ttttgagtca ttctttccat |
| 301 | gtggaattca aaggagaatg taaacaaatt ttcaggaggg acgtgcaata tccctgaaag |
| 361 | ataacaaagt tcgtaacact tatttacata caacattctc tagttattga ttaaacagat |
| 421 | ctctacagac ttgcatgagg caacatttct taggcttgtt tgctacaata tctttaaaaa |
| 481 | tacttgatta cacatcactt tagcttattt agATGgactt ttcaccaagc tctgaactgg |
| 541 | gatttcattt tgttgcattc atcctgctca cgagacacag gacagccttt cctgcctcag |
| 601 | ggaagaagag agagacagac tacagtgatg gagacccact agatgtgcac aagaggctgc |
| 661 | catccagtgc tggagaggac cgagccgtga tgctggggtt tgccatgatg ggcttctcag |
| 721 | tcctaatgtt cttcttgctc ggaacaacca ttctaaagcc ttttatgctc agcattcaga |
| 781 | gagaagaatc gacctgcact gccatccaca cagatatcat ggacgactgg ctggactgtg |
| 841 | ccttcacctg tggtgtgcac tgccacggtc aggggaagta cccgtgtctt caggtgtttg |
| 901 | tgaacctcag ccatccaggt cagaaagctc tcctacatta taatgaagag gctgtccaga |
| 961 | taaatcccaa gtgcttttac acacctaagt gccaccaaga tagaaatgat ttgctcaaca |
| 1021 | gtgctctgga cataaaagaa ttcttcgatc acaaaaatgg aacccccttt tcatgcttct |
| 1081 | acagtccagc cagccaatct gaagatgtca ttcttataaa aaagtatgac caaatggcta |
| 1141 | tcttccactg tttattttgg ccttcactga ctctgctagg tggtgccctg attgttggca |
| 1201 | tggtgagatt aacacaacac ctgtccttac tgtgtgaaaa atatagcact gtagtcagag |
| 1261 | atgaggtagg tggaaaagta ccttatatag aacagcatca gttcaaactg tgcattatga |
| 1321 | ggaggagcaa aggaagagca gagaaatctt aagacggtgg ccaaattaaa gtgctggcct |
| 1381 | tcagatgtct gtgatttctg caactgagga cctaattatg cctgtctgca aactaataat |
| 1441 | gtaaaaggta ataattaaag tatcatattt tcatgtggga aaaaatttaa aa |

Human KCNMB3, transcript variant 4, is encoded by the following polypeptide sequence (NCBI Accession No. NP_{—}055222.3 and SEQ ID NO: 14):

MDFSPSSELGFHFVAFILLTRHRTAFPASGKKRETDYSDGDPLDVHKR
LPSSAGEDRAVMLGFAMMGFSVLMFFLLGTTILKPFMLSIQREESTCT
AIHTDIMDDWLDCAFTCGVHCHGQGKYPCLQVFVNLSHPGQKALLHYN
EEAVQINPKCFYTPKCHQDRNDLLNSALDIKEFFDHKNGTPFSCFYSP
ASQSEDVILIKKYDQMAIFHCLFWPSLTLLGGALIVGMVRLTQHLSLL
CEKYSTVVRDEVGGKVPYIEQHQFKLCIMRRSKGRAEKS

Human KCNMA1, transcript variant 1, is encoded by the following mRNA sequence (NCBI Accession No. NM_{—}001014797 and SEQ ID NO: 15):

| | |
|---|---|
| 1 | cgccaggtcg cgcacagcgc cccgagccca ggcgcctccc cgcccccctc ccgcgctccg |
| 61 | cggcggcggc ggcggcggca gcagtagcag caatatggct gttgatgggt gtttggggtg |
| 121 | gcgctggcgg cgggaggagc tcccccgagc ccctgcgccg gctgcccgtt gctagctATG |
| 181 | gcaaatggtg gcggcggcgg cggcggcagc agcggcggcg gcggcggcgg cggaggcagc |
| 241 | agtcttagaa tgagtagcaa tatccacgcg aaccatctca gcctagacgc gtcctcctcc |
| 301 | tcctcctcct cctcttcctc ttcttcttct tcctcctcct cttcctcctc gtcctcggtc |
| 361 | cacgagccca agatggatgc gctcatcatc ccggtgacca tggaggtgcc gtgcgacagc |
| 421 | cggggccaac gcatgtggtg ggctttcctg gcctcctcca tggtgacttt cttcgggggc |
| 481 | ctcttcatca tcttgctctg gcggacgctc aagtacctgt ggaccgtgtg ctgccactgc |
| 541 | gggggcaaga cgaaggaggc ccagaagatt aacaatggct caagccaggc ggatggcact |
| 601 | ctcaaaccag tggatgaaaa agaggaggca gtggccgccg aggtcggctg gatgacctcc |
| 661 | gtgaaggact gggcgggggt gatgatatcc gcccagacac tgactggcag agtcctggtt |
| 721 | gtcttagtct ttgctctcag catcggtgca cttgtaatat acttcataga ttcatcaaac |
| 781 | ccaatagaat cctgccagaa tttctacaaa gatttcacat tacagatcga catggctttc |
| 841 | aacgtgttct tccttctcta cttcggcttg cggtttattg cagccaacga taaattgtgg |
| 901 | ttctggctgg aagtgaactc tgtagtggat ttcttcacgg tgccccccgt gtttgtgtct |
| 961 | gtgtacttaa acagaagttg gcttggtttg agatttttaa gagctctgag actgatacag |
| 1021 | ttttcagaaa ttttgcagtt tctgaatatt cttaaaacaa gtaattccat caagctggtg |
| 1081 | aatctgctct ccatatttat cagcacgtgg ctgactgcag ccgggttcat ccatttggtg |
| 1141 | gagaattcag gggacccatg ggaaaatttc caaaacaacc aggctctcac ctactgggaa |
| 1201 | tgtgtctatt tactcatggt cacaatgtcc accgttggtt atggggatgt ttatgcaaaa |
| 1261 | accacacttg ggcgcctctt catggtcttc ttcatcctcg ggggactggc catgtttgcc |
| 1321 | agctacgtcc ctgaaatcat agagttaata ggaaaccgca agaaatacgg gggctcctat |
| 1381 | agtgcggtta gtggaagaaa gcacattgtg gtctgcggac acatcactct ggagagtgtt |
| 1441 | tccaacttcc tgaaggactt tctgcacaag gaccgggatg acgtcaatgt ggagatcgtt |
| 1501 | tttcttcaca acatctcccc caacctggag cttgaagctc tgttcaaacg acattttact |
| 1561 | caggtggaat tttatcaggg ttccgtcctc aatccacatg atcttgcaag agtcaagata |
| 1621 | gagtcagcag atgcatgcct gatccttgcc aacaagtact gcgctgaccc ggatgcggag |
| 1681 | gatgcctcga atatcatgag agtaatctcc ataaagaact accatccgaa gataagaatc |
| 1741 | atcactcaaa tgctgcagta tcacaacaag gcccatctgc taaacatccc gagctggaat |
| 1801 | tggaaagaag gtgatgacgc aatctgcctc gcagagttga agttgggctt catagcccag |
| 1861 | agctgcctgg ctcaaggcct ctccaccatg cttgccaacc tcttctccat gaggtcattc |
| 1921 | ataaagattg aggaagacac atggcagaaa tactacttgg aaggagtctc aaatgaaatg |
| 1981 | tacacagaat atctctccag tgccttcgtg ggtctgtcct tccctactgt ttgtgagctg |
| 2041 | tgttttgtga agctcaagct cctaatgata gccattgagt acaagtctgc caaccgagag |
| 2101 | agccgaagcc gaaagcgtat attaattaat cctggaaacc atcttaagat ccaagaaggt |
| 2161 | actttaggat ttttcatcgc aagtgatgcc aaagaagtta aaagggcatt tttttactgc |
| 2221 | aaggcctgtc atgatgacat cacagatccc aaaagaataa aaaaatgtgg ctgcaaacgg |
| 2281 | cttgaagatg agcagccgtc aacactatca ccaaaaaaaa agcaacggaa tggaggcatg |
| 2341 | cggaactcac ccaacacctc gcctaagctg atgaggcatg accccttgtt aattcctggc |
| 2401 | aatgatcaga ttgacaacat ggactccaat gtgaagaagt acgactctac tgggatgttt |
| 2461 | cactggtgtg cacccaagga gatagagaaa gtcatcctga ctcgaagtga agctgccatg |
| 2521 | accgtcctga gtggccatgt cgtggtctgc atctttggcg acgtcagctc agccctgatc |
| 2581 | ggcctccgga acctggtgat gccgctccgt gccagcaact ttcattacca tgagctcaag |
| 2641 | cacattgtgt ttgtgggctc tattgagtac ctcaagcggg aatgggagac gcttcataac |
| 2701 | ttccccaaag tgtccatatt gcctggtacg ccattaagtc gggctgattt aagggctgtc |
| 2761 | aacatcaacc tctgtgacat gtgcgttatc ctgtcagcca atcagaataa tattgatgat |
| 2821 | acttcgctgc aggacaagga atgcatcttg gcgtcactca acatcaaatc tatgcagttt |
| 2881 | gatgacagca tcggagtctt gcaggctaat tcccaagggt tcacacctcc aggaatggat |
| 2941 | agatcctctc cagataacag cccagtgcac gggatgttac gtcaaccatc catcacaact |
| 3001 | ggggtcaaca tccccatcat cactgaacta gtgaacgata ctaatgttca gtttttggac |
| 3061 | caagacgatg atgatgaccc tgatacagaa ctgtacctca cgcagccctt tgcctgtggg |
| 3121 | acagcatttg ccgtcagtgt cctggactca ctcatgagcg cgacgtactt caatgacaat |
| 3181 | atcctcaccc tgatacggac cctggtgacc ggaggagcca cgccggagct ggaggctctg |
| 3241 | attgctgagg aaaacgccct tagaggtggc tacagcaccc cgcagacact ggccaatagg |
| 3301 | gaccgctgcc gcgtggccca gttagctctg ctcgatgggc catttgcgga cttaggggat |
| 3361 | ggtggttgtt atggtgatct gttctgcaaa gctctgaaaa catataatat gctttgtttt |
| 3421 | ggaatttacc ggctgagaga tgctcacctc agcaccccca gtcagtgcac aaagaggtat |
| 3481 | gtcatcacca acccgcccta tgagtttgag ctcgtgccga cggacctgat cttctgctta |
| 3541 | atgcagtttg accacaatgc cggccagtcc cgggccagcc tgtcccattc ctcccactcg |
| 3601 | tcgcagtcct ccagcaagaa gagctcctct gttcactcca tcccatccac agcaaaccga |
| 3661 | cagaaccggc ccaagtccag ggagtcccgg gacaaacaga acagaaaaga aatggtttac |
| 3721 | agatgaaccg gataatgcct atcccagaaa cattcaaatc aagcccatga gtacccacat |
| 3781 | ggctaaccag atcaaccaat ataaatccac aagcagcttg attccaccaa tcagagaagt |
| 3841 | tgaagatgaa tgttgactcc caggagacca gaactatttt tttaaagcct gacaaacttc |
| 3901 | ataaatggtg atgtgacttt tcttcttaca tgctgaatca ctggtggaaa cgttaggata |
| 3961 | agcaagaatt gccagaaaat aaagtagaca gatctttctc tttgtctgcc ttgaatattg |
| 4021 | cttccatgta ttttggaaaa gaaaatgatt tcatttataa atgaacatac taaaatagtc |
| 4081 | atgtatagcc tctagcattt taaattattt ttatttatta gaaagaaaat atattttttc |
| 4141 | ttttcattgt caaatatctt ccctatatct aaacaatgca aaatctaaat gaataagtgg |
| 4201 | tcagactcct taatgtgttt ttctcttctt tctctctttt tctttgagga gaatgatggt |
| 4261 | caccaccaca attaattgta attaagggaa atgaattatt aaaacaattt taaaatggcc |
| 4321 | aatgatgtat acatgtattt tagtaaatcc agaaaagaag actaagggga caggtgaata |
| 4381 | cctgctcctc gcctggatgg gtgtgtattt tgatctgcat tgacaccagc tcttaataaa |
| 4441 | tggaaactta tttattttca cagttgaaag tcatattttt gtagttctag ttttcattct |
| 4501 | gtagttctca cccctttgtt catattttta aagggaagac cttctgctgc ttttctacaa |
| 4561 | ttggcagatc aggtttgtct ggccaactaa ggacaccagg agggttgtgg gcacacctgg |
| 4621 | gatgtggaag ctgaggacca gatgcacaca aggcattgct ctgtttgctg ttctagtggt |
| 4681 | ggagactcct acgtgctgtt atgcttctca ttttatattt ccaaaccacc tgagcgagta |
| 4741 | atgtcaactt cgggagggtc tggtggactt tctccccact ccccaagtct cagagctttt |
| 4801 | agcctggaag tttgtaaaac tgattagtag ttttacttta ccataggatg tgagagggca |
| 4861 | gtcattcttc tcactgagtt tttgtttaaa aaccaggaaa tttctacaat catggagaca |
| 4921 | gattttaatt tgcatgctct cctttattta gttgctgggg ccaaacacat gaacttgtta |
| 4981 | caacaattta aaaataataa taacaaaagg aaagcaacga atgcacttca tatagactgt |
| 5041 | tagaaaaggc ctttgaaaat taccagcagt gaaatcaaat aaattagcca ctttaaactg |
| 5101 | cggtcaaaag atttttctag ccatcagatg caattacgat gtctcagagc tctgctatcg |
| 5161 | cttgcatgct cggtgtacag tctcccggca actgttttgt tccaatcaac ttagcaagtc |
| 5221 | tttcaacttt aaaattatca atctttgata tgatcatcaa ggtctctgga gaagcatgga |
| 5281 | tgtaatagtg atttctattt tcttaaaaaa tgtaaactgt ggacacttaa tggaaccaag |
| 5341 | ctaaccaaag tatctccccg aagtttcatg atatctagtc cattaactct ttgggccatg |
| 5401 | acctgtggta cctagttatg gtagtgcaaa tcagatcaga gcaaagcaat gagatcatca |
| 5461 | tgaagacata tgtccactaa gtactccaat taccagcgta ggcaaaatga gcttccacct |
| 5521 | ctgtccactc cctgcagagc caatatagtc gtgcttccac ctcgtaggca ttgtcctgca |
| 5581 | gtgatttgtc ggactgtggt tactctgtcg tcacattttg tagaattagt tctataaaag |
| 5641 | tattgtgaat acaatgtatg tccaattgga ttgtttaaaa actacttata attaatcact |
| 5701 | agcctactca gcagactaaa atgatgtcaa cagtctatat tcaggcttat aaacatttct |
| 5761 | tggtttccaa cagaagtcaa atgtgtttgt ggggagaacg ttttgctagg attttgacaa |
| 5821 | ctttcctgaa agctaggcct tttttacata atgattttgc attggggtca ccttagtata |
| 5881 | ttcacgtcaa tccacgctct tctttccttc gtgaaccata gtgggctctt cactcctgct |
| 5941 | cgtaataacc acctccaaat ttgttagagt gatttggtct cctctggcag gctgtggtgg |
| 6001 | ccacataaag gatcttccat tagagggaca caggacacaa agctccattc ccatgaccct |
| 6061 | ctcttccctg tgtctcccct aacaaggcaa tctggaagca gaaatgcaaa ctccagcccc |
| 6121 | accacacatc caccgatgca tttgtggcta gaaattttaa tctaaacttt ttttaaaaaa |
| 6181 | aatcataata gttaggatag tttcctatag caaagggctc cttgattcat aagcacctcc |
| 6241 | cccccaaaaa aaccaaattg gtaaaaattc attttctttt aaatagagca ataagttcat |
| 6301 | actgtgttgc cgttgtaata atgaattgtt ctggactaga aaacaaaatg aggcattttg |
| 6361 | ttaagggggt aggaaaatga ctgattgttg ggccctttgg caaaagctgg tgcctggtaa |
| 6421 | gtagaaacga aatggaaaca gaaataacat ttgtgaaatc actggagggc tcaagcaaat |
| 6481 | gccagagctt ctccctggct tgttcctact tgttgagctc tttggccctg agtcccatcc |
| 6541 | atctccactg caacagtctg tctttctgcc tacatccgcc tgagaccaag gagggtcttt |
| 6601 | ggtacagtcc acattatgag gctctgattt cctatcccag tttagggctc cagaaaagcc |
| 6661 | attctcccaa actaatgatt cttgggtgct gtgggtcacc aacaggctgt ttacaaagca |
| 6721 | gaaaatatca tcaccatcat aacctttctc cttgagaaaa cttttatttt tgtctaagca |
| 6781 | attttatatt ttagaatatc atcttgaacc attataaaac accttaaata aaagtgattg |
| 6841 | tgactaaggc acgttggaga acattccaat ctttcctccc aagggttctg gcaccttatg |
| 6901 | atgtactttg taaaaaaatt gattgaagat tttaaataaa caggaaatta gaagtgttag |
| 6961 | ggttagataa aggaaaagtg aaaaagcata agaaatttgg gactttttca tgttgaaagg |
| 7021 | agaaaaagca caagttccct agttaccatt taatatgagg gtgcaagagc cacatttaca |
| 7081 | aacaacaaac ttgatcttta aaaatgtttt taaaactctc tgagtctcag tgacttcatt |
| 7141 | tgtaaatgag agtgattcat ctagatgact ctaaggtctc tttcaggtcc caggggacgt |
| 7201 | gatctaagtt aggagtggga acatgggagt ggctgcttct ccttggctaa gcccatgcag |
| 7261 | actattgtgt ccaggactgg aataaaggtc ctttttgctg catcacccag gcaacactcc |
| 7321 | cagccttact caggagtggt gaacattttc tgctctggac atagaatagg taggttcatt |
| 7381 | cctggtcttt tcactttaga ttctctttag gttcaagatg cagatttctc aatttggaca |
| 7441 | cacctgcatc acgttggtca aattttgctg cctgtttcta gggttttctt ctcagtttca |
| 7501 | tccctgcctt cccttttttg ttgttttttt tttggttcaa ggcccagctt cagttaatcc |
| 7561 | cttcatctct atgccatgtc tccaattcct agtatgtaac tagaggttca agagacccca |
| 7621 | aattcctgcc attttcccca tttatgtttc cccatgtgaa tcttcaggaa gtaaccagtc |
| 7681 | ttccttctca cttcatttcc tgtcctctgt ttatttggtt attttctttc attaataaac |
| 7741 | taaccatctc ttagctactt acattcacaa atgtaatttt aatctattgt gtttcattgt |
| 7801 | gaaagagaat ctcattgaag gaaggaagga agaaattgag gaataaaggt aggaagggag |
| 7861 | agaaacaaag actatccaac caattgactg agatgtgagc cggcttgact cctaggagac |
| 7921 | taagtagatc cattcagctt gaacactgag ttccagggca gaatctgtcc attttctgtt |
| 7981 | ttgttcttac ttgctgcttt tggaatggtg gtaaagccac cagtcaccat gagccttgaa |
| 8041 | aggaccttca ctaatgagga cttgactcac cctctttaaa aagcaaaaga aacagaattt |
| 8101 | acttacaatc ttctggtaaa ttacctttct gatcctctcc ttattcccaa aaggcaattt |
| 8161 | ttttaatcca gcagagatga tttagcatat aaatcccagg tcatttgagg aggtgatgac |
| 8221 | gctatttgga tttatttatg agacagtagt aattataaat gattcctagg cagtttgtac |
| 8281 | tagtttggtc atattggttg tttaagtgga gtctcttctg attgtttgtt tccaataaag |
| 8341 | tatcagttgt taataatttc aagatttcca taaaaataag aaaacttgga aaacattttt |
| 8401 | aattttattt cttaagagac cacagagcct aattttcaat ttttatggta agcttgctta |
| 8461 | aaacatgtat aaaaagaagc ctacatgctc aaaggcagag gcacctttag ccctacaaca |
| 8521 | gttggaagct ccatgatatc acagccccac atggatagat tggagaaaga attctgactt |
| 8581 | agaccacctg tagcagacct tcctcttttg gcatattttt ttaatcacaa aaaggacctg |
| 8641 | agactcccta tctgaagata agccaaactt ggggtataca ttaagaatct gggaagaaag |
| 8701 | aaaaaaaaga aaaagatata ataagcagta ttttaaaata ccatgagtta tgggcatgca |
| 8761 | tatggtacaa agagtagttc tttgggttga aagaaagaag agtttgcatt gaactgcgta |
| 8821 | ggctggcaag aatgcagagg tatacaatgt ttgatgggct ttggaggcat ttcaatagta |
| 8881 | aatgggtgac aggactggaa agatgtggaa tatgagggtt ttccagggag atgggactta |
| 8941 | atgggccatg atgtatttgt aggaatgtga tttcaataga ttgactattt tgtctactgt |
| 9001 | gataagtttg tgtaggggaa ctgaggaaaa ctgagatgag acttcatgga aggggctaca |
| 9061 | aagtacagtc taaaggccta gataaagttt gaaacttgat ttagaaagtc atgaaaagcc |
| 9121 | actcagagtt ttaagcaaag agtgatgggg cagcaatggc tgtgcatttt tcaggttgaa |
| 9181 | agtagtgaga tgtcagttct tcctggaaga tatcatgaga atgcacacaa aactgaacat |
| 9241 | tttctcagca gcaaggagtg ctaaagggtg cacactccaa gtcaacaact caccgtgtgc |
| 9301 | agcaccttcc tcacacattt tgctttgctt tctgtcacag gaccatcaga cgtagaacgt |
| 9361 | cctgactctc ctgaattact agaatgtcag caacaaactc acaaagtaga ttagagggta |
| 9421 | ggaatagata cataaatttt taaaaacctg ccaatcatac tgttttctag acagtaatat |
| 9481 | aataagaaca tgaggcagga catccacaaa tagtagagaa taattaatag tccagaactg |
| 9541 | ccaaatagga attcccctta tgaaaacacc cttgcagcta atcaggtagc ccttgtttag |
| 9601 | aaaccaatta gcactcataa aatatttgta caaggagtga gtgggggaga aatgagcaca |
| 9661 | atatgggttc tgccatcaat cagcttataa tacaactaaa gaaagaaaac atgctcaaag |
| 9721 | caccattaga gaaagggtga gccctaatga catagtttca cattcaatat ggtaggagtt |
| 9781 | gaaacaaggg agaaatgctg tggtttgaag tggttcaaca aggtaggatt tctagagaaa |
| 9841 | ggggggttta ggagttagat aagtagagga tcagcattct aggtatgaag gcaggattat |
| 9901 | ctggcatgtg gcagggtgaa tgatgttaat cagtaactgc taattgatat ttactgagca |
| 9961 | gctgctgtgt cctcacgatg gtaagactat tagaggaaga cagcccacct gcctaactgg |
| 10021 | gaacacaagg cacacatata ttggaaagat catgttgcac aaaacaggtg ttcaataaat |
| 10081 | aatttttgaa taaatgtgaa cactgaatag caaataaatg ttatagatcg taatagttgt |
| 10141 | agaagattgg ttgggattgt agggactgga tgtggggcaa tggctgtggg tctagagtat |
| 10201 | attcattgtg cagtggacac acacctaatc tgtcctcaag gacagaacca tgtggaggaa |
| 10261 | taggaaaaca agagaagcag gcaagaagcg gaggcagtga gtacagcagc taagaaatgt |
| 10321 | tacattgggt tcatccagag caagaaaagc tgagagctgt ccgtgaggga aggccacaca |
| 10381 | ggcaagagga gtgggacagg ggagtaaagg agtgggctac aacaggccct gcagatgtgg |
| 10441 | aagccagacc cacagaggca cttagaaagt gaggatcctg gatgaattat tttagggaga |
| 10501 | aagtgtaggt ttagatcagc tggcagatgt gtttgtgggc tacagccaag tttttcagga |
| 10561 | ctaagaagga gcaaaataag tagaacttct tgggacagga gatctagggt actccagaac |
| 10621 | cagggaaatg agaaacaaga ggggaatttt aagaaagaga gggaagatca tctatgaggt |
| 10681 | tggagatgaa aggtgttgga gatggtgagg cacaagtcaa aagactcact cgaagggcag |
| 10741 | gaactctgct tatttgtcca tattcccaga gtttgtcatg aggtctggcc cacgtgggca |
| 10801 | gtcactcggc cactatgtat cactgtggct gccatggtgg atggaggctg gacagaacct |
| 10861 | gtggtgatcg gcagtctttc tggaggactc accagggagg ggtctcaggg tgggcatctc |
| 10921 | ctttcacctt cctgtgcatg tcactgcact gttcttcttc cctccctgaa cagcacacac |
| 10981 | actgcatcct tagtttcagg ggtctgaaaa caaatgctac gaccaccttg ctttggtggc |
| 11041 | ttttctgaac aggcattgtg agaagagatg ttctggccag gtgaaagtgg actttatttt |
| 11101 | taggtaggtt tttcagttta gaaaggtttc ctttgcttat cccttgagga cttctgtgct |
| 11161 | gctgcttctc ctccatcttg ctagttaaca gaacaggggc agaagtgaca ccaatgctct |
| 11221 | cggccatgcc tgccttggga agcactaccg ggccccactg tttagaatct tagatggaaa |
| 11281 | tgtacacaag gcattgacac acgcctgcgg gagtgaccca gtgcttccct cccatgggac |
| 11341 | tcagggtcag cgtgggaagg cagctggcag ggacaggagc cccgcccgca aggtggactg |
| 11401 | agcctcactt ccacacaaaa ccagagtccc atctgcccca gagcgctcgc tctcctcccc |
| 11461 | ttccttccgc tcaatgcagc ctgacacgcc tttgaccaga actgcaacat ttcaaacaca |
| 11521 | aacccagatc tgttgggaaa tcctaattaa aaacgttgca ttcccaggtg gcacaatcac |
| 11581 | atcctgccta aaacttctgg cccacatccc accacccaga tgttgaaaag aaacctttct |
| 11641 | ctgcttaaca ccaaaacctg cacttcaagg tttctttagg ggacaaagaa aaaaaaagta |
| 11701 | cacacatgca gagttcagcc tacatgttca ttgaggaaaa agagcgtggc ttattcatct |
| 11761 | ttcaatcttt tgattgttgg cattatgatt atgattatga ttaattactc tgacctgaca |
| 11821 | ggaattgaag aagagactat atgctggtgc tctgaaatga tctacccaac tctgtcatct |
| 11881 | gtaacaacca gtgataactc tcttgtcttg taaaaagggt ttgtacataa cttgtacatg |
| 11941 | gtttcatttt gtatttttcg cagattaaaa atttatgtat ttgtgttcta aaa |

Human KCNMA1, transcript variant 1, is encoded by the following polypeptide sequence (NCBI Accession No. NP_{—}001014797.1 and SEQ ID NO: 16):

MANGGGGGGGSSGGGGGGGGSSLRMSSNIHANHLSLDASSSSSSSSSS
SSSSSSSSSSSSVHEPKMDALIIPVTMEVPCDSRGQRMWWAFLASSMV
TFFGGLFIILLWRTLKYLWTVCCHCGGKTKEAQKINNGSSQADGTLKP
VDEKEEAVAAEVGWMTSVKDWAGVMISAQTLTGRVLVVLVFALSIGAL
VIYFIDSSNPIESCQNFYKDFTLQIDMAFNVFFLLYFGLRFIAANDKL
WFWLEVNSVVDFFTVPPVFVSVYLNRSWLGLRFLRALRLIQFSEILQF
LNILKTSNSIKLVNLLSIFISTWLTAAGFIHLVENSGDPWENFQNNQA
LTYWECVYLLMVTMSTVGYGDVYAKTTLGRLFMVFFILGGLAMFASYV
PEIIELIGNRKKYGGSYSAVSGRKHIVVCGHITLESVSNFLKDFLHKD
RDDVNVEIVFLHNISPNLELEALFKRHFTQVEFYQGSVLNPHDLARVK
IESADACLILANKYCADPDAEDASNIMRVISIKNYHPKIRIITQMLQY
HNKAHLLNIPSWNWKEGDDAICLAELKLGFIAQSCLAQGLSTMLANLF
SMRSFIKIEEDTWQKYYLEGVSNEMYTEYLSSAFVGLSFPTVCELCFV
KLKLLMIAIEYKSANRESRSRKRILINPGNHLKIQEGTLGFFIASDAK
EVKRAFFYCKACHDDITDPKRIKKCGCKRLEDEQPSTLSPKKKQRNGG
MRNSPNTSPKLMRHDPLLIPGNDQIDNMDSNVKKYDSTGMFHWCAPKE
IEKVILTRSEAAMTVLSGHVVVCIFGDVSSALIGLRNLVMPLRASNFH
YHELKHIVFVGSIEYLKREWETLHNFPKVSILPGTPLSRADLRAVNIN
LCDMCVILSANQNNIDDTSLQDKECILASLNIKSMQFDDSIGVLQANS
QGFTPPGMDRSSPDNSPVHGMLRQPSITTGVNIPIITELVNDTNVQFL
DQDDDDDPDTELYLTQPFACGTAFAVSVLDSLMSATYFNDNILTLIRT
LVTGGATPELEALIAEENALRGGYSTPQTLANRDRCRVAQLALLDGPF
ADLGDGGCYGDLFCKALKTYNMLCFGIYRLRDAHLSTPSQCTKRYVIT
NPPYEFELVPTDLIFCLMQFDHNAGQSRASLSHSSHSSQSSSKKSSSV
HSIPSTANRQNRPKSRESRDKQNRKEMVYR

Human KCNMA1, transcript variant 2, is encoded by the following mRNA sequence (NCBI Accession No. NM_{—}002247 and SEQ ID NO: 17):

| | |
|---|---|
| 1 | cgccaggtcg cgcacagcgc cccgagccca ggcgcctccc cgcccccctc ccgcgctccg |
| 61 | cggcggcggc ggcggcggca gcagtagcag caatatggct gttgatgggt gtttggggtg |
| 121 | gcgctggcgg cgggaggagc tcccccgagc ccctgcgccg gctgcccgtt gctagctATG |
| 181 | gcaaatggtg gcggcggcgg cggcggcagc agcggcggcg gcggcggcgg cggaggcagc |
| 241 | agtcttagaa tgagtagcaa tatccacgcg aaccatctca gcctagacgc gtcctcctcc |
| 301 | tcctcctcct cctcttcctc ttcttcttct tcctcctcct cttcctcctc gtcctcggtc |
| 361 | cacgagccca agatggatgc gctcatcatc ccggtgacca tggaggtgcc gtgcgacagc |
| 421 | cggggccaac gcatgtggtg ggctttcctg gcctcctcca tggtgacttt cttcgggggc |
| 481 | ctcttcatca tcttgctctg gcggacgctc aagtacctgt ggaccgtgtg ctgccactgc |
| 541 | gggggcaaga cgaaggaggc ccagaagatt aacaatggct caagccaggc ggatggcact |
| 601 | ctcaaaccag tggatgaaaa agaggaggca gtggccgccg aggtcggctg gatgacctcc |
| 661 | gtgaaggact gggcgggggt gatgatatcc gcccagacac tgactggcag agtcctggtt |
| 721 | gtcttagtct ttgctctcag catcggtgca cttgtaatat acttcataga ttcatcaaac |
| 781 | ccaatagaat cctgccagaa tttctacaaa gatttcacat tacagatcga catggctttc |
| 841 | aacgtgttct tccttctcta cttcggcttg cggtttattg cagccaacga taaattgtgg |
| 901 | ttctggctgg aagtgaactc tgtagtggat ttcttcacgg tgccccccgt gtttgtgtct |
| 961 | gtgtacttaa acagaagttg gcttggtttg agatttttaa gagctctgag actgatacag |
| 1021 | ttttcagaaa ttttgcagtt tctgaatatt cttaaaacaa gtaattccat caagctggtg |
| 1081 | aatctgctct ccatatttat cagcacgtgg ctgactgcag ccgggttcat ccatttggtg |
| 1141 | gagaattcag gggacccatg ggaaaatttc caaaacaacc aggctctcac ctactgggaa |
| 1201 | tgtgtctatt tactcatggt cacaatgtcc accgttggtt atggggatgt ttatgcaaaa |
| 1261 | accacacttg ggcgcctctt catggtcttc ttcatcctcg ggggactggc catgtttgcc |
| 1321 | agctacgtcc ctgaaatcat agagttaata ggaaaccgca agaaatacgg gggctcctat |
| 1381 | agtgcggtta gtggaagaaa gcacattgtg gtctgcggac acatcactct ggagagtgtt |
| 1441 | tccaacttcc tgaaggactt tctgcacaag gaccgggatg acgtcaatgt ggagatcgtt |
| 1501 | tttcttcaca acatctcccc caacctggag cttgaagctc tgttcaaacg acattttact |
| 1561 | caggtggaat tttatcaggg ttccgtcctc aatccacatg atcttgcaag agtcaagata |
| 1621 | gagtcagcag atgcatgcct gatccttgcc aacaagtact gcgctgaccc ggatgcggag |
| 1681 | gatgcctcga atatcatgag agtaatctcc ataaagaact accatccgaa gataagaatc |
| 1741 | atcactcaaa tgctgcagta tcacaacaag gcccatctgc taaacatccc gagctggaat |
| 1801 | tggaaagaag gtgatgacgc aatctgcctc gcagagttga agttgggctt catagcccag |
| 1861 | agctgcctgg ctcaaggcct ctccaccatg cttgccaacc tcttctccat gaggtcattc |
| 1921 | ataaagattg aggaagacac atggcagaaa tactacttgg aaggagtctc aaatgaaatg |
| 1981 | tacacagaat atctctccag tgccttcgtg ggtctgtcct tccctactgt ttgtgagctg |
| 2041 | tgttttgtga agctcaagct cctaatgata gccattgagt acaagtctgc caaccgagag |
| 2101 | agccgtatat taattaatcc tggaaaccat cttaagatcc aagaaggtac tttaggattt |
| 2161 | ttcatcgcaa gtgatgccaa agaagttaaa agggcatttt tttactgcaa ggcctgtcat |
| 2221 | gatgacatca cagatcccaa aagaataaaa aaatgtggct gcaaacggct tgaagatgag |
| 2281 | cagccgtcaa cactatcacc aaaaaaaaag caacggaatg gaggcatgcg gaactcaccc |
| 2341 | aacacctcgc ctaagctgat gaggcatgac cccttgttaa ttcctggcaa tgatcagatt |
| 2401 | gacaacatgg actccaatgt gaagaagtac gactctactg ggatgtttca ctggtgtgca |
| 2461 | cccaaggaga tagagaaagt catcctgact cgaagtgaag ctgccatgac cgtcctgagt |
| 2521 | ggccatgtcg tggtctgcat ctttggcgac gtcagctcag ccctgatcgg cctccggaac |
| 2581 | ctggtgatgc cgctccgtgc cagcaacttt cattaccatg agctcaagca cattgtgttt |
| 2641 | gtgggctcta ttgagtacct caagcgggaa tgggagacgc ttcataactt ccccaaagtg |
| 2701 | tccatattgc ctggtacgcc attaagtcgg gctgatttaa gggctgtcaa catcaacctc |
| 2761 | tgtgacatgt gcgttatcct gtcagccaat cagaataata ttgatgatac ttcgctgcag |
| 2821 | gacaaggaat gcatcttggc gtcactcaac atcaaatcta tgcagtttga tgacagcatc |
| 2881 | ggagtcttgc aggctaattc ccaagggttc acacctccag gaatggatag atcctctcca |
| 2941 | gataacagcc cagtgcacgg gatgttacgt caaccatcca tcacaactgg ggtcaacatc |
| 3001 | cccatcatca ctgaactagt gaacgatact aatgttcagt ttttggacca agacgatgat |
| 3061 | gatgaccctg atacagaact gtacctcacg cagccctttg cctgtgggac agcatttgcc |
| 3121 | gtcagtgtcc tggactcact catgagcgcg acgtacttca atgacaatat cctcaccctg |
| 3181 | atacggaccc tggtgaccgg aggagccacg ccggagctgg aggctctgat tgctgaggaa |
| 3241 | aacgccctta gaggtggcta cagcaccccg cagacactgg ccaataggga ccgctgccgc |
| 3301 | gtggcccagt tagctctgct cgatgggcca tttgcggact taggggatgg tggttgttat |
| 3361 | ggtgatctgt tctgcaaagc tctgaaaaca tataatatgc tttgttttgg aatttaccgg |
| 3421 | ctgagagatg ctcacctcag cacccccagt cagtgcacaa agaggtatgt catcaccaac |
| 3481 | ccgccctatg agtttgagct cgtgccgacg gacctgatct tctgcttaat gcagtttgac |
| 3541 | cacaatgccg gccagtcccg ggccagcctg tcccattcct cccactcgtc gcagtcctcc |
| 3601 | agcaagaaga gctcctctgt tcactccatc ccatccacag caaaccgaca gaaccggccc |
| 3661 | aagtccaggg agtcccggga caaacagaag tacgtgcagg aagagcggct ttgatatgtg |
| 3721 | tatccaccgc cactgtgtga aactgtatct gccactcatt tccccagttg gtgtttccaa |
| 3781 | caaagtaact ttccctgttt tcccctgtag tccccccctt tttttttaca catatttgca |
| 3841 | tatgtatgat agtgtgcatg tggttgtcat ttttatttca ccaccataaa acccttgagc |
| 3901 | acaacagcaa ataagcagac ggaccaaaag ttatttatga ttctggggga aaaataaccc |
| 3961 | aaaggcatgc tccagacata aatagctcac tgcaggaacg agttcacaga ttagaaggga |
| 4021 | gcacttgtga tcaacgtcag ttaggcagag caagtttatt taatgtaaaa gaaaagttga |
| 4081 | ttctgattta tcaggattat cagggtgctt tgggttttga ttttgttgtt gttgttgttt |
| 4141 | tcctttcttt ctttttttat acacacaata agttagcaca tgtttatttg aaacaagcaa |
| 4201 | ccaaacagca atgaaaacat attgattgtt tccagtctct gggccgaagt attgcgaagc |
| 4261 | atttgaaaag ctttcacgat ttgtgtagat gattatgaag gacctgcttg ttgcaagaga |
| 4321 | acatcagtga tttttttagt tactcaccaa ggccttttgt cccagagcca gttccctctg |
| 4381 | ggagttctta tgaacatttc tcaccttaat atggaggaga gaatagtatt ccaatcatgg |
| 4441 | atgtatcaaa ttctagtcat ttagtttaag tgaaaagagg tttgattgca tattaaattg |
| 4501 | ttattctgtc tccttatgtt gccatatgaa tagctatttt ttttctttca cttttgacat |
| 4561 | ttgggatgaa aagccatatg tatcataaat atcagatgta agtcattaaa aactgccttc |
| 4621 | ctgggacttt tacatctttt aaaaggtgaa ttacttacct tatgtacaga ataaataatg |
| 4681 | ctcaggaaag agcaagtatt tttccatgca ttctcagggg atctttttac tcccctttgt |
| 4741 | ttgattagtt agggccccaa tgccaggtag gaggaagggc tggggcaatg gtagagtgag |
| 4801 | aggaagacaa acccagctgc agatcatgct tttctaggag ccgacatgct aaataaatta |
| 4861 | gaatgtagga ggatcagcca cagttgactc aacaaagaca aaagccagcc accaccttca |
| 4921 | actgttggca cagctgtgcg gtgctggctg tcccaatgca gaaagctggt gggaaggaat |
| 4981 | tcctcatcat cactttcttt aatgtagcca atttaggcag ggtaatgacg gcaatagaga |
| 5041 | gctgctcctt gtcattatga gacgtgggat aagaagagtg caacagtgag ccaaacacat |
| 5101 | tttggtatag ttattttttt cttcttttgt tttctttctt ttttaacact tagtaagcat |
| 5161 | gagaggagag gtagaaaaat accctttttt caacatatag ttgtcagatg ctttgtgcat |
| 5221 | gcaaatcatg ctttaggcag tgcggtattt cttaaaaact ggccaattca ccataaccaa |
| 5281 | tttcccttat ggatggacta ggctggtata tacatatttg aaaagtttta cttcaaagaa |
| 5341 | ttccatcgaa tagaataggg gtaaaaggga ggaggaaaac atgtcacagc tgtaccatct |
| 5401 | ctaaaaaggt gtttttatgg tgaatgtttt ggatttagat tttggatccc ccgtcccctc |
| 5461 | aagcatgata gttttggata tttgcttgct gtgtgaattg acaagcactt ttactgacaa |
| 5521 | atggtgaggc tcagtcagaa cctccaccct cccccacacc aaagacaggg gcagcgtagt |
| 5581 | attcaaacca gtattgtggt ggggaataat tgtatacatg taaattatca agccctatga |
| 5641 | gtggaagaat tttttcaaat tatttttgtc cctctatata ttgatttata ttatgtataa |
| 5701 | ctatctcttt atataaacta tatataatta tatatatata actatataat tatatatata |
| 5761 | taactatata tataactata tatatgtatc ccctagtatt ggatcatgaa gagctcttca |
| 5821 | tgcattcttt gcaaaggagg ttataaagtt acgccctcag aacatttata actataagaa |
| 5881 | tgtgccagtt aaagtgctca acaggaaata tgacagttta aaagcattgt aaaactcaca |
| 5941 | tagcttactt ctctctctaa agtgcaacaa ggatgaatag aatgggccaa ggtatgacaa |
| 6001 | ttaatggttc tgcatgacct agccactgct gggggttttc ttctataacg ttgtccttgt |
| 6061 | gaaaactttt gtgaaattaa aaaaaaagga gttacaaatt tta |

Human KCNMA1, transcript variant 2, is encoded by the following polypeptide sequence (NCBI Accession No. NP_{—}002238.2 and SEQ ID NO: 18):

MANGGGGGGGSSGGGGGGGGSSLRMSSNIHANHLSLDASSSSSSSSSS
SSSSSSSSSSSSVHEPKMDALIIPVTMEVPCDSRGQRMWWAFLASSMV
TFFGGLFIILLWRTLKYLWTVCCHCGGKTKEAQKINNGSSQADGTLKP
VDEKEEAVAAEVGWMTSVKDWAGVMISAQTLTGRVLVVLVFALSIGAL
VIYFIDSSNPIESCQNFYKDFTLQIDMAFNVFFLLYFGLRFIAANDKL
WFWLEVNSVVDFFTVPPVFVSVYLNRSWLGLRFLRALRLIQFSEILQF
LNILKTSNSIKLVNLLSIFISTWLTAAGFIHLVENSGDPWENFQNNQA
LTYWECVYLLMVTMSTVGYGDVYAKTTLGRLFMVFFILGGLAMFASYV
PEIIELIGNRKKYGGSYSAVSGRKHIVVCGHITLESVSNFLKDFLHKD
RDDVNVEIVFLHNISPNLELEALFKRHFTQVEFYQGSVLNPHDLARVK
IESADACLILANKYCADPDAEDASNIMRVISIKNYHPKIRIITQMLQY
HNKAHLLNIPSWNWKEGDDAICLAELKLGFIAQSCLAQGLSTMLANLF
SMRSFIKIEEDTWQKYYLEGVSNEMYTEYLSSAFVGLSFPTVCELCFV
KLKLLMIAIEYKSANRESRILINPGNHLKIQEGTLGFFIASDAKEVKR
AFFYCKACHDDITDPKRIKKCGCKRLEDEQPSTLSPKKKQRNGGMRNS
PNTSPKLMRHDPLLIPGNDQIDNMDSNVKKYDSTGMFHWCAPKEIEKV
ILTRSEAAMTVLSGHVVVCIFGDVSSALIGLRNLVMPLRASNFHYHEL
KHIVFVGSIEYLKREWETLHNFPKVSILPGTPLSRADLRAVNINLCDM
CVILSANQNNIDDTSLQDKECILASLNIKSMQFDDSIGVLQANSQGFT
PPGMDRSSPDNSPVHGMLRQPSITTGVNIPIITELVNDTNVQFLDQDD
DDDPDTELYLTQPFACGTAFAVSVLDSLMSATYFNDNILTLIRTLVTG
GATPELEALIAEENALRGGYSTPQTLANRDRCRVAQLALLDGPFADLG
DGGCYGDLFCKALKTYNMLCFGIYRLRDAHLSTPSQCTKRYVITNPPY
EFEINPTDLIFCLMQFDHNAGQSRASLSHSSHSSQSSSKKSSSVHSIP
STANRQNRPKSRESRDKQKYVQEERL

### The E65K Polymorphism

The human β-regulatory subunit gene includes a non-synonymous polymorphism that converts a glutamic acid to a lysine reside at codon 65 (KCNMB1-E65K) associated with the single nucleotide polymorphism (SNP) rs11739136. The E65K polymorphism (also referred to as the “K allele”) increases the probability of BK channel opening in response to calcium stimulation. With greater calcium responsiveness, BK channels that comprise the E65K mutant protein were found to increase vascular smooth muscle cell relaxation leading to greater vasorelaxation and lower blood pressure, including reduced diastolic blood pressure.

Genetic variants that influence large conductance calcium-activated potassium channel's function may alter arterial function and contribute to the known heritability of arterial stiffness and blood pressure. The β1-subunit (KCNMB1) of the large conductance calcium-activated potassium channel includes two coding region polymorphisms. E65K, a gain-of-function polymorphism, was predicted to enhance large conductance calcium-activated potassium channel opening and vasorelaxation, whereas V110L has no known effect. This mechanism was tested in humans. The data is consistent with the mechanism of E65K polymorphism reducing muscular artery stiffness. This effect was observed in muscular arteries that are relevant to blood pressure control.

To test whether E65K has a favorable effect on arterial function, arterial tonometry and brachial artery phenotypes were examined relative to genotypes in 1100 Framingham Offspring Study participants with available genotypes and phenotypes (53% women; mean age 61.5±9.4 years).

The minor allele frequency was 0.10 for E65K and 0.09 for V110L; both were in Hardy-Weinberg equilibrium (χ² P>0.05), and haplotype analysis found R²=0.01. E65K was associated with lower augmented pressure (7.4±3.3 versus 9.0±3.8 mmHg, P+0.01) and central pulse pressure (47.1±7.3 versus 50.7±7.8 mmHg, P=0.01) in multivariable analyses. No association was noted between E65K and mean arterial pressure, carotid-femoral pulse wave velocity or brachial artery diameter, flow velocity or volume flow. V110L was not associated with tonometry or brachial measures.

A diminished augmented pressure in K-carriers indicated a reduced or delayed wave reflection and supports the finding that E65K reduces arterial impedance mismatch in the arterial tree. The data indicated that this polymorphism increases the likelihood of the BK-channel being open in the vascular smooth muscle cell. As a consequence of greater probability of the channel being open, the polymorphism causes greater vascular smooth muscle cell relaxation. And as a consequence of greater cellular relaxation, the polymorphism leads to greater blood vessel relaxation, which in turn leads to lower blood pressure. Therefore, E65K polymorphism causes the blood pressure to be lower and has a favorable effect on arterial function and pulsatile hemodynamic load.

### Genotyping

The term “genotyping” refers to the process of determining the genotype of an individual. Standard, art-recognized, methods include, but are not limited to, polymerase chain reaction (PCR), DNA sequencing, and hybridization of sample genetic material to microarrays or beads. The term “single nucleotide polymorphism (SNP)” refers to a DNA sequence variation occurring when a single nucleotide in genomic DNA differs between individuals or between paired chromosomes within an individual. The term “allele” refers to a DNA region that occupies a given locus, or position, on a chromosome. Alleles are sequences that often code for a gene. The genotype of an individual for any given gene comprises a combination of two alleles that correlate to sequences found on either chromosome of the diploid pair. For example, an individual can have two copies of a mutation, one wild type and one mutant copy, or two wild type copies of a gene. The number of mutant copies can indicate the severity of a condition.

In one embodiment of the present invention, the choice of antihypertensive therapy varies according to the number of mutations carried by that individual. For example, an individual carrying two mutant alleles receives a different antihypertensive treatment than an individual carrying one mutant allele. Alternatively, an individual carrying two mutant alleles receives a different dose of the same antihypertensive treatment given to an individual carrying one mutant allele.

Subjects' genomic DNA was extracted from peripheral blood leukocytes using standard methods. The KCNMB1 polymorphism rs11739136 was genotyped using Taqman Assay (ABP Prism 7900HT; Applied Biosystems), using primers: 5′ ggtactggggcaccttcttgcccttcagct 3′ and 3′ ctcctggtccctgatgttggtctcaatcag 5′. Given the low frequency of the K-allele, the assay was validated by sequence confirmation (ABI Prism Big Dye Terminator 3.0, Applied Biosystems). Genotyping rates were 98.7% for Heart SCORE and 99.6% for FHS.

### Study Population: Heart SCORE Cohort

Heart SCORE is a prospective, community study based in Western Pennsylvania and designed to enroll equal numbers of Caucasian and African American participants. Subjects were enrolled at baseline; eligibility criteria included age 45 to 74 years, residence in the greater Pittsburgh metropolitan area, ability to undergo baseline and annual follow-up visits, and absence of known co-morbidity expected to limit life expectancy to less than 5 years. Among the 2,000 subjects enrolled at baseline, 1,191 had DNA available for analysis.

Assessment of demographics, past medical history, medication use, and physical examination were documented at the baseline enrollment visit. Physical examination included measurement of blood pressure and anthropometric measures of height and weight. Blood samples were drawn to measure fasting levels of glucose and other cardiovascular risk factors. Laboratory tests were performed using standard techniques in the clinical laboratory. For the present study, Heart SCORE subjects with history of cardiovascular disease were excluded, leaving n=1075 available for analysis.

### Study Population: Framingham Heart Study

The FHS Offspring cohort is a community-based study of healthy Caucasian-Americans that has previously been described in detail (Kannel W B et al. Am J. Epidemiol. 1979; 110:281-90). Among the 3,370 unrelated participants who attended the sixth clinic examination (1996-1998), analysis was restricted to 1,635 subjects with DNA available.

Medical history, medication use, laboratory tests and physical examinations were documented at examination cycle six. Systolic and diastolic blood pressure values were averaged from two physician-obtained measurements. Subjects with a history of cardiovascular disease were excluded leaving n=1519 for analysis.

### Blood Pressure-Lowering Drugs

One or more blood pressure-lowering drugs that are used individually or combinatorially to treat hypertension. Exemplary drugs include, but are not limited to, diuretics, angiotensin-coverting enzyme (ACE) inhibitors, angiotensin-2 receptor antagonists, beta blockers, calcium channel blockers, alpha blockers, and antiandrenergics.

Beta blockers are also referred to as beta-adrenergic blocking agents, beta-adrenergic antagonists, or beta antagonists. Beta blockers inhibit the action of endogenous catecholamines on β-adrenergic receptors. Exemplary catecholamines include, but are not limited to, epinephrine (also known as adrenaline) and norepinephrine (also known as noradrenaline). β-adrenergic receptors comprise a component of the sympathic nervous system which mediates the commonly named “flight or fight” response.

The present invention comprises beta blockers that target and/or inhibit the action of any β-adrenergic receptor. Exemplary receptors include β₁-, β₂-, and β₃-adrenergic receptors. Selective and non-selective beta blockers comprise the present invention. Exemplary non-selective agents include, but are not limited to, alprenolol, carteolol, levobunolol, metipreanolol, nadolol, oxprenolol, penbutolol, pindolol, propanolol, sotalol, and timolol. Exemplary β₁-selective agents include, but are not limited to, acebutolol, atenolol, betoxolol, bisoprolol, esmolol, metoprolol, and nebivolol. Exemplary β₂-selective agents include, but are not limited to, butaxamine. Some beta blockers also antagonize α₁-adrenegic receptors providing additional arteriolar vasodilation. Exemplary mixed α₁/β-adrenergic antagonists include, but are not limited to, carvedilol, celiprolol, and labetalol.

Diuretics include, but are not limited to, Amiloride (Midamor®), Bumetanide (Bumex®), Chlorothiazide (Diuril®), Chlorthalidone (Chlorthalidone Tablets®, Hygroton®, Thalitone®), Ethacrynic Acid (Edecrin®), Furosemide (Lasix®), Hydrochlorothiazide (Aldactazide®, Capozide®, Dyazide®, Esidrix®, HydroDIURIL®, Inderide®, Lopressor® HCT, Maxzide®, Microzide®, Moduretic®, Timolide®, Vaseretic® and combination drugs), Indapamide (Lozol®), Metolazone (Mykrox®, Zaroxolyn®), Spironolactone (Aldactone®, Aldactazide® as a combination product also containing Hydrochlorthiazide), Torsemide (Demadex Oral®), and Triamterene (Dyrenium®).

Angiotensin-coverting enzyme (ACE) inhibitors include, but are not limited to, Benazepril (Lotensin®), Captopril (Capoten®), Enalapril (Vasotec®), Fosinopril (Monopril®), Lisinopril (Prinivil®, Zestril®), Moexipril (Univasc®), Perindopril (Aceon®), Quinapril (Accupril®), Ramipril (Altace®), and Trandolapril (Mavik®).

Combinations of ACE inhibitors and diuretics are encompassed by the invention. Exemplary combinations include, but are not limited to, Benazepril and hydrochlorothiazide (Lotensin® HCT), Enalapril and hydrochlorothiazide (Vaseretic®), Fosinopril and hydrochlorothiazide (Monopril® HCT), Lisinopril and hydrochlorothiazide (Prinzide®, Zestoretic®), Moexipril and hydrochlorothiazide (Uniretic®), and Quinapril and hydrochlorothiazide (Accuretic®).

Angiotensin-2 receptor antagonists include, but are not limited to, Candesartan (Atacand®), Eprosartan (Teveten®), Irbesartan (Avapro®), Losartan (Cozaar®), Telmisartan (Micardis®), and Valsartan (Diovan®).

Combinations of angiotensin-2 receptor antagonists and diuretics are encompassed by the invention. Exemplary combinations include, but are not limited to, Eprosartan and hydrochlorothiazide (Teveten® HCT), Irbesartan and hydrochlorothiazide (Avalide®), Losartan and hydrochlorothiazide (Hyzaar®), Telmisartan and hydrochlorothiazide (Micardis® HCT), and Valsartan and hydrochlorothiazide (Diovan® HCT).

Beta blockers comprise: 1) Non-selective beta blockers that block both the Beta-1 and Beta-2 adrenergic receptors, including, but not limited to, Carteolol (Cartrol®), Nadolol (Corgard®, Corzide® as a combination product also containing Bendroflumethiazide), Propranolol (Inderal®, Innopran® XL, Propranolol Hydrochloride Intensol®); 2) Beta blockers that selectively block the Beta-1 adrenergic receptors, including, but not limited to, Atenolol (Tenormin®), Betaxolol Hydrochloride (Kerlone®), Bisoprolol (Zebeta®), Metoprolol (Lopressor®, Toprol XL®), Timolol (Blocadren®); 3) Beta blockers with intrinsic sympathomimetic activity, including, but not limited to, Acebutolol (Sectral®), and 4) Combination Beta and Alpha Adrenergic Receptor blocker activity, including, but not limited to, Carvedilol (Coreg®).

Beta blockers include, but are not limited to, Acebutolol (Sectral®), Atenolol (Tenormin®), Betaxolol Hydrochloride (Kerlone®), Bisoprolol (Zebeta®), Carteolol (Cartrol®), Metoprolol (Lopressor®, Toprol XL®), Nadolol (Corgard®, Corzide® as a combination product also containing Bendroflumethiazide), Penbutolol (Levatol®), Pindolol (Visken®), Propranolol (Inderal®, Innopran® XL, Propranolol Hydrochloride Intensol®), Sotalol (Betapace®, Sorine®), and Timolol (Blocadren®).

Combinations of beta blockers and diuretics are encompassed by the invention. Exemplary combinations include, but are not limited to Atenolol and Chlorthalidone (Tenoretic®), Bisoprolol and Hydrochlorothiazide (Ziac®), Propranolol and Hydrochlorothiazide (Inderide®), and Timolol and Hydrochlorothiazide (Timolide®).

Calcium channel blockers include, but are not limited to, Amlodipine (Norvasc®, Caduet® as a combination product also containing Atorvastatin for cholesterol), Bepridil (Vascor®), Diltiazem (Cardizem®, Cartia XT®, Dilacor XR®, Diltia XT®, Tiazac®), Felodipine (Plendil®), Isradipine (DynaCirc®), Nifedipine (Adalat®, Nifedical® XL, Procardia®), Nimodipine (Nimotop®), Nisoldipine (Sular®), and Verapamil (Calan®, Covera-HS®, Isoptin®, Verelan®).

Combinations of ACE inhibitors and calcium channel blockers are encompassed by the invention. Exemplary combinations include, but are not limited to, Amlodipine and benazepril (Lotrel®), Enalapril and felodipine (Lexxel®), and Trandolapril and verapamil (Tarka®).

Alpha blockers include, but are not limited to, Doxazosin mesylate (Cardura®), Prazosin hydrochloride (Minipress®), Prazosin and polythiazide (Minizide®), and Terazosin hydrochloride (Hytrin®).

Central alpha agonists, either given individually or combinatorially, include, but are not limited to, Clonidine hydrochloride (Catapres®, Duraclon®), Clonidine hydrochloride and chlorthalidone (Clorpres®), Guanabenz Acetate (Wytensin®), Guanfacine hydrochloride (Tenex®), Methyldopa (Aldomet®), Methyldopa and chlorothiazide (Aldochlor®), and Methyldopa and hydrochlorothiazide (Aldoril®).

Combinations of alpha and beta blockers are encompassed by the invention. Exemplary combinations include, but are not limited to, Carvedilol (Coreg®) and Labetalol hydrochloride (Normodyne®, Trandate®).

Adrenergics include, but are not limited to, Hylorel®.

### Statistical Analysis

served genotype frequencies were compared with those expected under Hardy-Weinberg equilibrium (HWE) using a χ2 test. Given the low prevalence of minor alleles, a dominant mode of inheritance was used in all analyses (E=wild type, K=substitution). Analyses were performed separately by race, and Caucasian genotype frequencies were similar in both cohorts. Analysis of covariance (ANOVA) was used to assess whether E65K was associated with adjusted mean systolic, diastolic and pulse pressure. Similarly, logistic regression was used to estimate the adjusted odds of being in the upper quartile of systolic, diastolic, or pulse pressure in relation to the E65K polymorphism. Models controlled for known and suspected blood pressure confounders, including current anti-hypertensive treatment. The interaction between E65K with anti-hypertensive medications, age and gender were tested a priori by the addition of a multiplicative term to the full multivariate model.

Analysis using pooled cohorts provides greater statistical power than independent cohort validation. The direction of the E65K-effect was similar in both cohorts; therefore, Caucasians were pooled in order to increase statistical power in order to detect a genetic interaction. All analyses were performed using SAS software (SAS Institute, Inc., Cary, N.C.). A two-sided p-value of <0.05 was considered statistically significant for all primary and secondary analyses. No adjustment for multiple testing was performed.

### EXAMPLES

### Example 1

### E65K Polymorphism and Blood Pressure in Heart SCORE Study

An association between the E65K polymorphism and blood pressure was tested in the Heart SCORE (n=1,075). Study cohorts composed of individuals who are not diagnosed with cardiovascular disease.

In Heart SCORE Caucasians (n=687), but not African-Americans (n=388), analysis of covariance revealed an association between E65K and lower systolic (E=wild type, K=substitution) (adjusted-mean mmHg (+SD) for E:133.7 (±7.3), K 130.6 (±8.4), p=0.02) and diastolic pressures (E 79.7 (±2.6), K 77.6 (±3.1), p=0.02). In logistic regression, Caucasian K-carriers were at lower risk for elevated systolic (OR=0.55, 95% CI: 0.33-0.91) and diastolic (OR=0.47, 95% CI: 0.28-0.79) blood pressure. The protective effect of E65K was primarily evident among subjects receiving anti-hypertensive therapy.

### Example 2

### E65K Polymorphism and Blood Pressure in Framingham Heart Study

An association between the E65K polymorphism and blood pressure was tested in the Framingham Heart Study (n=1,519) cohorts composed of individuals who are not diagnosed with cardiovascular disease.

In the Framingham Heart Study, the E65K polymorphism was not associated with blood pressure or hypertension, although a similar trend was noted. In pooled Caucasian Heart SCORE and Framingham Heart Study subjects we found that in subjects receiving beta-blockade, K-carriers had lower systolic and diastolic pressure (p=0.09, 0.03, respectively). In summary, Caucasian wild-type carriers are less responsive to beta-blockade treatment than subjects harboring the E65K allele.

### Example 3

### E65K Interacts with Age and Blood Pressure Treatment

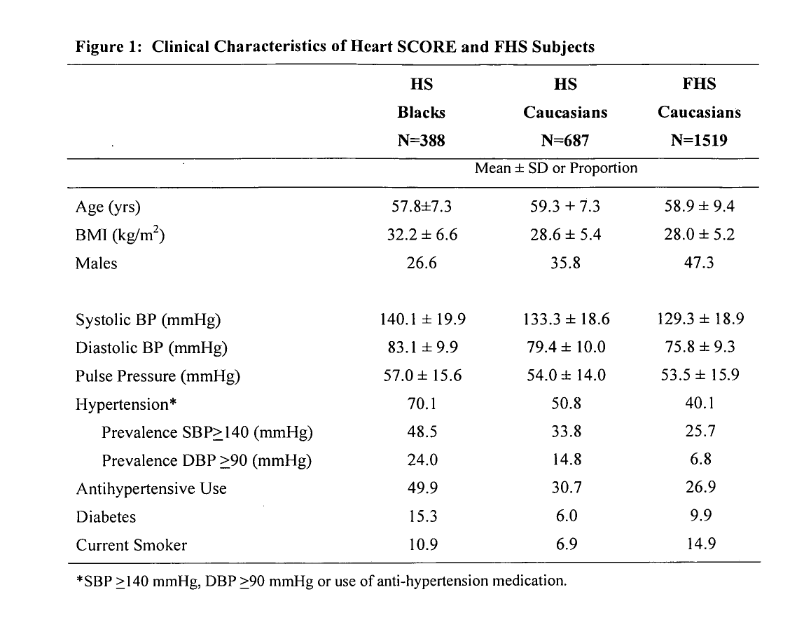

To evaluate whether E65K interacts with and antihypertensive therapy, age and gender, data from the Heart SCORE Caucasian and FHS cohorts was pooled to maximize power for detection. No interaction with gender was found. In adults receiving antihypertensive therapy, K-carriers had significantly lower adjusted SBP and DBP (p=0.03, 0.01, respectively) solely in the setting of anti-hypertensive treatment (FIG. 1). There was no difference in subjects not receiving antihypertensive therapy (p=0.27, 0.76, for SBP, DBP respectively). Testing for interaction between E65K and anti-hypertensive therapy was significant (p=0.049 DBP model).

There was no difference in the frequency of antihypertensive therapy use by E65K genotype (p=0.28). The distribution of major anti-hypertensive classes in treated subjects (n=619) was as follows: 54.6% reported receiving diuretics (n=338); 40.2% reported use of renin-angiotensin-system blocking agents (n=249); 37.8% reported beta-blockade use (n=234); and 25.5% reported using calcium-channel blockers (n=158), with a significant proportion (44.7%, n=277) receiving multiple agents.

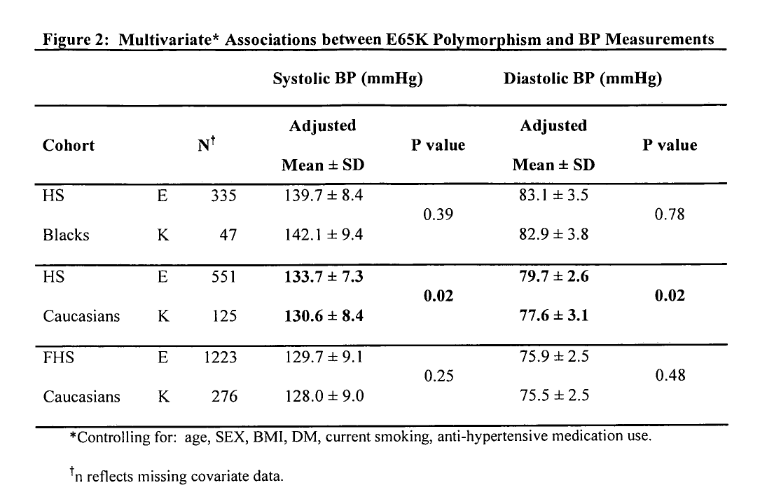

When differences in systolic and diastolic pressure by E65K status were examined by medication class, lower pressure was found amongst K-carriers receiving beta-blockade (FIG. 2), in all subjects and more pronounced in older subjects (age>58 years). No significant difference in blood pressure was noted by genotype status amongst renin-angiotensin-system blockade, calcium-channel blockers or diuretic classes (Table 4). Given that nearly 50% pf subject reported using multiple classes of antihypertensive medications, the analysis was limited to the use of a single agent, and results were similar (FIG. 2). Formal tests for interaction between beta-blockade and E65K genotype were not significant (p=0.29 and 0.15, SBP and DBP models, respectively). There was no difference in beta-blockade use by E65K status (p=0.28).

### Example 4

### The KCNMB1 E65K Carrier Status Identifies Patients with Hypertension that Respond Favorably to Beta Blocker Treatment

The relationship between the coding non-synonymous polymorphism (E65K) in the KCNMB1 gene and blood pressure in two independent community-based cohorts without cardiovascular disease was examined. Heart SCORE Caucasian K-allele carriers exhibited lower systolic and diastolic pressure, however, these effects were not found in Framingham Heart Study participants. This association is driven by the response in K-carriers receiving beta-blockade therapy.

The increased calcium-triggered BK activity of the mutant β₁-subunit protein preserves BK channel-dependent vasodilation in the setting of reduced overall (β₁-subunit expression. This effect was observed to be preferential in the setting of beta-blockade. Alternatively, the E65K polymorphism lowers blood pressure by modifying vascular remodeling or sensitivity to antihypertensive therapy in the setting of hypertension.

### Example 5

### Additional Polymorphisms in BK Channel Components

While it is possible that other KCNMB1 SNPs or haplotypes may have a stronger blood pressure association, E65K was tested for several reasons. First, only the E65K polymorphism has a biological function. Second, polymorphism screening of the KCNMB1 gene has not identified additional functional polymorphisms. Third, the KCNMB1 gene has a relatively low level of linkage disequilibrium.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of selecting an anti-hypertensive therapy for an individual in need thereof comprising testing for the presence or absence of a non-synonymous substitution within the nucleic acid or amino acid sequence of a wild type BK channel protein, wherein a therapeutic treatment is selected based on the presence of said substitution.

2. The method of claim 1, wherein said protein is KCNMB1.

3. The method of claim 1, wherein said protein is KCNMA1.

4. The method of claim 1, wherein said genetic mutation leads to an amino acid substitution at position 65 in the KCNMB1 protein.

5. The method of claim 1, wherein said genetic mutation is a substitution of a lysine (K) for a glutamic acid (E) residue at amino acid position 65 (E65K) in the KCNMB1 protein.

6. The method of claim 1, wherein said individual is identified as carrying a E65K substitution and wherein said anti-hypertensive therapy comprises a beta blocker.

7. The method of claim 1, wherein said anti-hypertensive therapy is selected from the group consisting of a diuretic, an angiotensin-coverting enzyme (ACE) inhibitor, an angiotensin-2 receptor antagonist, a beta blocker, a calcium channel blocker, an alpha blocker, and an antiandrenergic.

8. The method of claim 1, wherein said anti-hypertensive therapy comprises a combination of one or more anti-hypertensive therapies of claim 4.

9. The method of claim 1, wherein said anti-hypertensive therapy does not comprise verapamil.

10. The method of claim 1, wherein said hypertension condition is primary or secondary hypertension.

11. The method of claim 1, wherein said BK channel protein comprises a transcript variant of KCNMA1, KCNMB1, KCNMB2, or KCNMB3.

12. The method of claim 1, wherein said BK channel protein comprises a protein isoform of KCNMA1, KCNMB1, KCNMB2, or KCNMB3.

13. The method of claim 1, wherein said BK channel protein comprises a transcript variant or protein isoform of KCNMB1.

14. The method of claim 6, wherein said beta blocker comprises an inhibitor of β₁-, β₂-, and β₃-adrenergic receptors.

15. The method of claim 6, wherein said beta blocker is a selective or non-selective beta blocker.

16. The method of claim 6, wherein said beta blocker is selected from the group consisting of alprenolol, carteolol, levobunolol, metipreanolol, nadolol, oxprenolol, penbutolol, pindolol, propanolol, sotalol, and timolol.

17. The method of claim 6, wherein said beta blocker is selected from the group consisting of acebutolol, atenolol, betoxolol, bisoprolol, esmolol, metoprolol, and nebivolol.

18. The method of claim 6, wherein said beta blocker is selected from the group consisting of butaxamine, carvedilol, celiprolol, and labetalol.
